(19) United States
(12) Patent Application Publication
(10) Pub. No.: US 2008/0045608 A1
(43) Pub. Date: Feb. 21, 2008

(54) Gene Encoding a Guanine Nucleotide Exchange Factor Binding to Rhoa

(75) Inventors: Osamu Ohara, Chiba (JP); Takahiro Nagase, Chiba (JP); Michio Ohishi, Chiba (JP); Hiroshi Yokota, Tokyo (JP); Osamu Kamida, Tokyo (JP)

Correspondence Address: KILYK & BOWERSOX, P.L.L.C., 400 HOLIDAY COURT, SUITE 102, WARRENTON, VA, 20186, US

(73) Assignees: DAIICHI PHARMACEUTICAL CO., LTD. TOKYO R&D CENTER, Tokyo (JP); KAZUSA DNA RESEARCH INSTITUTE FOUNDATION, Kisarazu-shi (JP)

(21) Appl. No.: 11/578,326

(22) PCT Filed: Apr. 20, 2005
(86) PCT No.: PCT/JP05/07526

§ 371 Date: Nov. 21, 2006

(30) Foreign Application Priority Data
Apr. 21, 2004 (JP) 2004-125997

(51) Int. Cl. A61K 35/00; A61P 35/00; C07H 21/00; C07K 14/00; C07K 16/00; C12N 1/00; C12N 15/63; C12N 5/00; C12P 21/00; C12Q 1/68; G01N 33/68

(52) U.S. Cl. 514/789; 435/243; 435/320.1; 435/325; 435/6; 435/69.1; 436/86; 436/94; 530/350; 530/387.9; 536/23.1

(57) ABSTRACT

A gene encoding a novel protein that binds to a Rho family protein being one group of small GTP binding proteins and is capable of exhibiting a GEF activity, namely, a polynucleotide shown by the nucleotide sequence set forth in SEQ ID NO: 1, or SEQ ID NO: 3, or the complementary strand, the equivalents of the polynucleotide, a protein encoded by the polynucleotide, a vector containing the polynucleotide, a transformant containing the vector, an antibody against the protein encoded by the polynucleotide, a method of identifying a compound that inhibits the function of the protein encoded by the polynucleotide and/or the expression of the polynucleotide, a method of determining a disease, a pharmaceutical composition, and a reagent kit are provided.

### TECHNICAL FIELD

The present invention relates to proteins capable of binding to Rho family proteins that are one group of small GTP binding protein and capable of exhibiting an activity of a guanine nucleotide exchange factor (GEF) for the Rho family proteins, and to a polynucleotide encoding the protein. Specifically, the present invention relates to a protein that binds to Rho family small GTP binding protein RhoA, a polynucleotide encoding the protein, a recombinant vector containing the polynucleotide, and a transformant being transfected with the recombinant vector. Further, the present invention relates to a method of producing the protein, and an antibody raised against the protein. Furthermore, the present invention relates to a method of identifying a compound that inhibits the function of the protein and/or the expression of the polynucleotide. Further, the present invention relates to a method of diagnosing tonsil tumors and/or bronchial tumors, comprising measuring the amount of expression of the polynucleotide. Furthermore, the present invention relates to an agent for preventing and/or treating tonsil tumors and/or bronchial tumors containing an inhibitor for the function of the protein and/or an inhibitor for the expression of the polynucleotide, and a method of preventing and/or treating tonsil tumors and/or bronchial tumors, comprising using an inhibitor for the function of the protein and/or an inhibitor for the expression of the polynucleotide. Further, the present invention relates to a reagent kit containing at least one of the following components: the protein, the polynucleotide, the recombinant vector, the transformant and the antibody.

### BACKGROUND OF INVENTION

A Rho family small GTP binding protein (hereinafter, may be simply referred to as a Rho family protein) belongs to one group of small GTP binding proteins (hereinafter, may be simply referred to as a small G protein). A small G protein works as a signal amplifier between a cell membrane receptor, and an effector participating in an intracellular signal transduction pathway. Further, the small G protein specifically binds to guanosine 5′-triphosphate (GTP) or guanosine 5′-diphosphate (GDP), and shows an enzyme activity of hydrolyzing the bound GTP to GDP. When an extracellular signaling substance binds to a cell membrane receptor, its signal is transduced to a small G protein, which subsequently leads to a reaction exchanging a GDP bound to the small G protein for an intracellular GTP (hereinafter, the reaction may be abbreviated to GDP/GTP exchange reaction). Consequently, an active small G protein that is a GTP binding form is generated. The active small G protein acts on the effector to amplify the signal. Then, the GTP binding form of small G protein hydrolyzes the bound GTP to GDP by its enzyme activity and thereby become inactivated. Thus, the small G protein works as a molecular switch in an intracellular signal transduction pathway by exchanging the guanine nucleotides.

Cdc42, Rac1, RhoA and the like, are known as Rho family proteins. Cdc42 regulates firopodia formation in a fibroblast. Rac1 regulates superoxide production in leukocytes and macrophages, while it regulates cell membrane ruffling and lamellipodia formation in fibroblasts. Further, Cdc42 and Rac1 are capable of activating the c-Jun N-terminal kinase signal transduction pathway. Thus, Rho family proteins are involved in various kinds of cell function by regulating the intracellular signal transduction. For example, cytoskeleton restructuring, cell adhesion, gene expression, and the like, are known as a Rho family protein-mediated cell function. Such functions mediated by Rho family proteins are considered to regulate morphogenesis during ontogeny, migration of leukocyte and the like, axon degeneration, tumor metastasis, and invasion.

Rho guanine nucleotide exchange factor (hereinafter, may be abbreviated as Rho-GEF) is a member of a family of proteins involved in the molecular switching of a Rho family protein. Rho-GEF can function to accelerate the GDP/GTP exchange reaction of a Rho family protein, and can thereby convert the Rho family protein from a GDP-bound inactive form to a GTP-bound active form. Rho-GEF plays an important role through this function, in regulating Rho family protein-mediated intracellular signal transduction. Hereinafter, to accelerate the GDP/GTP exchange reaction and thereby to convert the Rho family protein from a GDP-bound inactive form to a GTP-bound active form may be referred to as a GEF activity or Rho family protein activation.

Rho-GEF has a characteristic domain structure, such as a Dbl homology domain (hereinafter, may be abbreviated as DH domain) and a pleckstrin homology domain (hereinaer, may be abbreviated as PH domain). The DH/PH tandem structure is a typical domain structure for Rho-GEF. Hereinafter, the tandem structure of DH domain and PH domain may be referred to as DH/PH domain.

The DH/PH domain is an important domain participating in the Rho-GEF-mediated activation of a Rho family protein, and is considered to be an active domain of Rho-GEF. For example, it has been reported that a protein, which comprises a C-terminal region of the amino acid sequence of proto-Dbl, and contains a DH/PH domain, activated a Rho family protein (Non-Patent Reference 1). proto-Dbl is a prototype of Rho-GEF. Specifically, this report showed that a protein consisting of a C-terminal region within the entire amino acid sequence of proto-Dbl with 925 amino acids, which was generated by deletion of the N-terminal amino acid residues from the 1^{st} to the 497^{th}, activated a Rho family protein, and consequently participated in cellular transformation. From these facts, the activation of proto-Dbl is considered to be an oncogenic activation. Hereinafter, a protein that consists of the C-terminal region of proto-Dbl is referred to as an oncogenic-Dbl. It has been reported that oncogenic-Dbl bind to RhoA, Cdc42 and Rac1, and that they have a GEF activity for Cdc42 and RhoA while they do not exert a GEF activity to Rac1 (Non-Patent Reference 2).

Vav (Non-Patent Reference 3 and 4), ost (Non-Patent Reference 5), lbc (Non-Patent Reference 6), and the like, are known to be genes that encode proto-Dbl family proteins. These genes are known to be involved in cancer. Further, trio (Non-Patent Reference 7), kalirin (Non-Patent Reference 8), and the like, are reported to be genes that encode proteins working as Rho-GEF. A trio knocked-out mouse shows an abnormal skeletal muscle structure and an abnormal brain structure in embryogenesis. Kalirin is involved in axon formation in neurons. Thus, each protein working as Rho-GEF is involved in a cellular function particular to each kind of protein, and activates a different Rho family protein.

Literatures referred in the present description are listed hereunder.

Non-patent Reference 1: Bi, F. et al., Molecular and Cellular Biology, 2001, Vol. 21, p. 1463-1474

Non-patent Reference 2: Hart, M. J. et al., Journal of Biological Chemistry, 1994, Vol. 269, p. 62-65

Non-patent Reference 3: Katzav, S. et al., EMBO Journal, 1989, Vol. 8, p. 2283-2290

Non-patent Reference 4: Costello, P. S. et al., Proceedings of The National Academy of Sciences of The United States of America, 1999, Vol. 96, p. 3035-3040

Non-patent Reference 5: Horii, Y. et al., EMBO Journal, 1994, Vol. 13, p. 4776-4786

Non-patent Reference 6: Toksoz, D. et al., Oncogene, 1994, Vol. 9, p. 621-628

Non-patent Reference 7: O'Brien, S. P. et al., Proceedings of The National Academy of Sciences of The United States of America, 2000, Vol. 97, p. 12074-12078

Non-patent Reference 8: Penzes, P. et al., Journal of Neuroscience, 2001, Vol. 21, p. 8426-8434

### DISCLOSURE OF THE INVENTION

### Problem To Be Solved By The Invention

An object of the present invention is to provide a novel Rho-GEF, and a gene encoding the Rho-GEF. Further, the object of the present invention includes providing a recombinant vector that contains the gene, and a transformant transfected with the recombinant vector. Furthermore, an object of the present invention includes providing a method of producing the Rho-GEF, and an antibody recognizing the Rho-GEF. Further, an object of the present invention includes providing a method of identifying a compound that inhibits the function of the Rho-GEF and/or the expression of the gene. Furthermore, an object of the present invention includes providing a method of preventing and/or treating a disease due to the abnormal function of the Rho-GEF and/or the abnormal expression of the gene, a method of diagnosing the disease, and a reagent kit.

### Means For Solving The Object

The present inventors have concentrated their efforts to meet the aforementioned object. Then, the present inventors have discovered a novel gene having a DH/PH domain-coding region characteristic for Rho GEF, and have successfully obtained the gene. Further, the present inventors revealed experimentally that a protein encoded by the gene was bound to a Rho family protein, RhoA. Moreover, the present inventors found that the tissue expression of the gene in a case of tonsil squamous cell carcinoma and a case of bronchial adenoid cystic carcinoma is at least 2.5 times or more higher, and at least 1.5 times or more higher, respectively, compared to that in a respective normal tissue. The present invention has been thus achieved.

In various embodiments, the present invention relates to a polynucleotide shown by the nucleotide sequence set forth in SEQ ID NO: 1 or by the complementary nucleotide sequence, or a polynucleotide encoding a protein shown by the amino acid sequence set forth in SEQ ID NO: 2 or a polynucleotide shown by the complementary nucleotide sequence of the polynucleotide.

The present invention also relates to a polynucleotide selected from the following group, wherein the polynucleotide encodes a protein that binds to RhoA:
(i) a polynucleotide shown by a nucleotide sequence having a homology of at least 70% with the nucleotide sequence of the aforementioned polynucleotide,
(ii) a polynucleotide with a mutation or an induced mutation, such as deletion, substitution, addition of one or several nucleotides in the nucleotide sequence of the aforementioned polynucleotide, and
(iii) a polynucleotide that hybridizes to the aforementioned polynucleotide under stringent conditions.

The present invention further relates to a polynucleotide selected from the following group, wherein the polynucleotide encodes a protein having a guanine nucleotide exchange factor (GEF) activity for RhoA:
(i) a polynucleotide shown by a nucleotide sequence having a homology of at least 70% with the nucleotide sequence of the aforementioned polynucleotide,
(ii) a polynucleotide with a mutation or an induced mutation, such as deletion, substitution, addition of one or more nucleotides in the nucleotide sequence of the aforementioned polynucleotide, and
(iii) a polynucleotide that hybridizes to the aforementioned polynucleotide under stringent conditions.

The present invention still further relates to recombinant vectors containing any one of the aforementioned polynucleotides.

The present invention also relates to transformants that have been transfected with any of the aforementioned recombinant vectors.

The present invention further relates to transformants that have been transfected with any of the aforementioned recombinant vectors and recombinant vectors containing a polynucleotide encoding RhoA.

The present invention still further relates to a protein shown by the amino acid sequence set forth in SEQ ID NO: 2 in the sequence listing.

The present invention also relates to proteins encoded by any one of the aforementioned polynucleotides.

The present invention further relates to methods of producing the aforementioned proteins, comprising a step of culturing the aforementioned transformants.

The present invention still further relates to antibodies that recognize the aforementioned proteins.

The present invention also relates to a method of identifying a compound that inhibits the function of the aforementioned proteins, and/or the expression of any one of the aforementioned polynucleotides. The method can comprise detecting the presence, absence or change in the function and/or the expression of any of the aforementioned proteins or polynucleotides, under conditions where the interaction of a compound with the protein and/or the polynucleotide are allowed, and determining whether the compound inhibits the function of the protein and/or the expression of the polynucleotide.

The present invention further relates to the aforementioned identification method, wherein the function of the protein is a GEF activity for RhoA.

The present invention still further relates to a method of determining whether a tissue specimen derived from a human tonsil tissue is a tissue derived from a human tonsil tumor or not, comprising measuring an amount of expression of any one of the aforementioned polynucleotides in the tissue specimen.

The present invention also relates to the aforementioned determination method, wherein the method determines that the tissue specimen is a tissue derived from a human tonsil tumor where the amount of expression of any one of the aforementioned polynucleotides in the tissue specimen is at least 2.5 times or more higher than that in a control tissue derived from human normal tonsil.

The present invention further relates to a method of determining whether a tissue specimen derived from a human bronchial tissue is a tissue derived from a human bronchial tumor or not, comprising measuring an amount of expression of any one of the aforementioned polynucleotides in the tissue specimen.

The present invention still further relates to the aforementioned determination method, wherein the method determines that the tissue specimen is a tissue derived from a human bronchial tumor where the amount of expression of any one of the aforementioned polynucleotides in the tissue specimen is at least 1.5 times or more higher than that in a control tissue derived from human normal bronchia.

The present invention also relates to agents for preventing and/or treating tonsil tumors and/or bronchial tumors, comprising a compound that inhibits the function of the aforementioned proteins, and/or a compound that inhibits the expression of any one of the aforementioned polynucleotides, as an active ingredient.

The present invention further relates to methods of preventing and/or treating tonsil tumors and/or bronchial tumors, comprising using a compound that inhibits the function of the aforementioned proteins, and/or a compound that inhibits the expression of any one of the aforementioned polynucleotides.

The present invention still further relates to a reagent kit containing at least one of the aforementioned proteins, the aforementioned polynucleotides, the aforementioned recombinant vectors, the aforementioned transformants, and the aforementioned antibodies.

### Advantage of the Invention

The present invention can provide polynucleotides encoding novel proteins that bind to Rho family proteins, and proteins encoded by the polynucleotides. A present protein was bound to a Rho family protein, RhoA. The proteins have a DH/PH domain that is considered to be an active domain of Rho-GEF in the amino acid sequence. Thus, the present proteins can be considered to bind to Rho proteins, such as RhoA, and to exhibit a GEF activity.

The present invention can also provide recombinant vectors containing the polynucleotides, and transformants transfected with the recombinant vectors. Further, the present invention can provide methods of producing the proteins, and antibodies specific to the proteins. Furthermore, the present invention can provide methods of identifying a compound that inhibits the function of the proteins and/or the expression of the polynucleotides. Further, the present invention can provide methods of diagnosing a tumor disease such as tonsil tumors and bronchial tumors, comprising measuring an amount of expression of the polynucleotides. Furthermore, the present invention can provide agents for preventing and/or treating a tumor disease such as tonsil tumors and bronchial tumors, comprising an inhibitor of the function of the proteins and/or an inhibitor of the expression of the polynucleotides, as an active ingredient. Further, the present invention can provide methods of preventing and/or treating a tumor disease such as tonsil tumors and bronchial tumors, comprising using the inhibitor of the function of the proteins and/or the inhibitor of the expression of the polynucleotides. Furthermore, the present invention can provide a reagent kit comprising at least one of the following components: the protein; the polynucleotide; the recombinant vector; the transformant; and the antibody.

The present invention allows elucidation and regulation of the signal transduction pathway and cellular function which are mediated by Rho family proteins such as RhoA. Further, the present invention allows diagnosis, prevention and/or treatment of a disease due to an abnormal function of the present proteins and/or an abnormal expression of the present polynucleotides, for example, a tumor disease, more specifically, tonsil tumors and bronchial tumors.

### BRIEF DESCRIPTION OF THE DRAWINGS

FIG. 1 shows a band corresponding to binding of a protein encoded by sh06537-F to RhoA, that was detected (lane 2 in the upper panel) in a cell lysate prepared from a cell into which a sh06537-F expression vector was co-transfected with a RhoA expression vector. The sh06537-F expression vector allows the expression of a protein encoded by a polynucleotide shown by the nucleotide sequence set forth in SEQ ID NO: 1 in the sequence listing, as a C-terminal 3×FLAG-tagged protein. A pull down assay was used for measuring the binding. On the other hand, such a band was not detected (lane 3 in the upper panel) in a cell lysate prepared from a cell into which a vector constructed by using DNA, that consists of a partial sequence of sh06537 lacking a DH/PH domain coding region (sh06537-D), was co-transfected with a RhoA expression vector. Further, such a band was not detected (lane 1 in the upper panel) in a cell lysate prepared from a cell into which only a RhoA expression vector was transfected. The middle panel shows results of detecting a protein encoded by sh06537-F, or a protein encoded by sh06537-D, in each cell lysate sample, by SDS-PAGE using an anti-FLAG antibody. The lower panel shows results of detecting a GST-fusion RhoA in each cell lysate sample, by SDS-PAGE using an anti-GST antibody. The amount of the protein encoded by sh06537-F, or the protein encoded by sh06537-D, was almost the same in each cell lysate sample (lanes 2 and 3 in the middle panel). Further, the amount of RhoA was almost the same in each cell lysate sample (lanes 1 to 3 in the lower panel). (Example 3)

### DETAILED DESCRIPTION OF THE INVENTION

Embodiments of the present invention are explained in further detail below.

In the present invention, the term “polynucleotide” may be used as a generic term which includes the following: an isolated full-length DNA and/or RNA; a synthetic full-length DNA and/or RNA; an isolated DNA oligonucleotide and/or RNA oligonucleotide; or a synthetic DNA oligonucleotide and/or RNA oligonucleotide. Such DNA and/or RNA used herein comprise two or more nucleotides.

In the present invention, the term “protein” may be used as a generic term which includes the following: an isolated or a synthetic full-length protein; an isolated or a synthetic full-length polypeptide; and an isolated or a synthetic full-length oligopeptide. A protein, a polypeptide or an oligopeptide used herein comprises two or more amino acids. Hereinafter, an amino acid may be represented by a single letter or by three letters.

(Polynucleotide)

An aspect of the present invention relates to a novel polynucleotide. The present polynucleotide was identified and isolated from a cDNA library derived from human spleen tissue as a gene having a region encoding a DH/PH domain that is a characteristic domain for Rho-GEF.

The polynucleotide according to the present invention can be a polynucleotide shown by the nucleotide sequence set forth in SEQ ID NO: 1 in the sequence listing, or by the complementary nucleotide sequence thereof. The polynucleotide shown by the nucleotide sequence set forth in SEQ ID NO: 1 is 5541 bp long and contains an open reading flame (ORF) encoding 1597 amino acid residues (SEQ ID NO: 2). The region in the nucleotide sequence set forth in SEQ ID NO: 1, which consists of the nucleotides from the 3706^{th} to the 4245^{th}, encodes a DH domain consisting of 180 amino acid residues from the 1178^{th} valine (Val) to the 1357^{th} asparagine (Asn) of the amino acid sequence set forth in SEQ ID NO: 2. The region in the nucleotide sequence set forth in SEQ ID NO: 1, which consists of the nucleotides from the 4345^{th} to the 4680^{th}, encodes a PH domain consisting of 112 amino acid residues from the 1391^{st} leucine (Leu) to the 1502^{nd} glutamic acid (Glu) of the amino acid sequence set forth in SEQ ID NO: 2. The region in the nucleotide sequence set forth in SEQ ID NO: 1, which consists of the nucleotides from the 3706^{th} to the 4680^{th}, encodes a DH/PH domain consisting of 325 amino acid residues from the 1178^{th} valine (Val) to the 1502^{nd} glutamic acid (Glu) of the amino acid sequence set forth in SEQ ID NO: 2.

A polynucleotide encoding a protein shown by the amino acid sequence set forth in SEQ ID NO: 2, or a polynucleotide shown by the complementary nucleotide sequence of the polynucleotide is also included in the scope of the present invention.

The polynucleotide according to the present invention can be preferably a polynucleotide encoding a protein that binds to Rho family proteins, or a polynucleotide shown by the complementary nucleotide sequence of the polynucleotide. The phrase “binding between proteins” used in the present invention refers to the interaction of a certain protein (protein A) with an another certain protein (protein B) so as to form a complex by non-covalent bond such as hydrogen bond, hydrophobic bond, electrically static interaction or the like. The binding of protein A to protein B only at the portion of these molecules is enough to be referred as “the binding” mentioned herein. For example, an amino acid which is not involved in the binding of protein A to protein B may be contained in the amino acid that constitutes protein A or protein B.

The polynucleotide shown by the nucleotide sequence set forth in SEQ ID NO: 1 encodes a protein that was bound to a Rho family protein, RhoA. Concretely, in an animal cell into which the polynucleotide shown by the nucleotide sequence set forth in SEQ ID NO: 1 was co-expressed with a gene encoding RhoA, the binding of a protein encoded by the polynucleotide to RhoA was detected (See Example 3). On the other hand, in an animal cell into which the polynucleotide shown by the nucleotide sequence set forth in SEQ ID NO: 3 was co-expressed with a gene encoding RhoA, no binding was detected between a protein encoded by the polynucleotide and RhoA (See Example 3). The polynucleotide shown by the nucleotide sequence set forth in SEQ ID NO: 3 is a polynucleotide shown by the nucleotides from the 175^{th} nucleotide to the 3693^{rd} nucleotide of the nucleotide sequence set forth in SEQ ID NO: 1. The polynucleotide shown by the nucleotide sequence set forth in SEQ ID NO: 3 lacks a DH/PH domain coding region that is present in the polynucleotide shown by the nucleotide sequence set forth in SEQ ID NO: 1. Thus, the polynucleotide (SEQ ID NO: 3) lacking a DH/PH domain coding region encodes a protein that did not bind to RhoA. These findings revealed that a DH/PH domain is participating in the binding of a protein encoded by the polynucleotide shown by the nucleotide sequence set forth in SEQ ID NO: 1, to RhoA. A DH/PH domain is known to be important for the binding of Rho-GEF to a Rho family protein, and for a GEF activity of Rho-GEF.

The polynucleotides according to the present invention encode proteins that have a DH/PH domain in the structure, and bind to Rho family proteins. A DH/PH domain is important for the binding of Rho-GEF to a Rho family protein, and for a GEF activity of Rho-GEF. Therefore, the inventors believe that the polynucleotides according to the present invention encode proteins that bind to Rho family proteins and exhibit a GEF activity for the Rho family proteins. In other words, the present polynucleotides encode proteins that bind to Rho family proteins and activate the Rho family proteins.

The phrase “GEF activity for Rho family proteins” or “activation of Rho family proteins” means to exchange guanosine 5′-diphosphate (GDP), that is bound to a Rho family protein, for guanosine 5′-triphosphate (GTP), and thereby to convert the Rho family protein from a GDP-bound inactive form to a GTP-bound active form. This exchange reaction comprises a dissociation reaction of GDP from a Rho family protein, and a binding reaction of GTP to the resultant Rho family protein, without a nucleotide being bound. Specifically, the phrase “GEF activity for RhoA” or “activation of RhoA” means to convert RhoA from an inactive form to an active form via an exchange reaction of RhoA-bound GDP for intracellular GTP.

In the present specification, the phrase “GEF activity for Rho family proteins” and the phrase “activation of Rho family proteins” can be used interchangeable.

A Rho family protein, which is activated by the proteins encoded by the polynucleotides according to the present invention, may be preferably exemplified by RhoA. In the present invention, the phrase “Rho family protein” may preferably direct to RhoA. A Rho family protein is not limited to this specific example and can be any Rho family protein, as long as it is activated by the proteins encoded by the present polynucleotides. It can be preferably exemplified by Rho family proteins that are activated by being bound with the proteins encoded by the present polynucleotides. The binding of the proteins encoded by the present polynucleotides to Rho family proteins can be measured by using, for example, a pull-down assay (see Example 3). In addition, the activation of Rho family proteins by the proteins encoded by the present polynucleotides can be measured by using, for example, an effector pull-down assay, which is described later.

The amino acid sequence of RhoA and the nucleotide sequence of RhoA gene are set forth in SEQ ID NO: 9 and SEQ ID NO: 8, respectively. RhoA and its gene are not limited to the specific examples shown by the aforementioned sequences. They can be a protein or a gene with a mutation of one or several sites in the aforementioned sequence, as long as it has a function of RhoA generally known. Further, a mutant can be prepared for use by introducing a mutation into one or several sites of the aforementioned sequence in order to enhance or diminish the function of the protein and the gene. RhoA can be produced, for example, by culturing a transformant that was prepared by transfecting with a recombinant vector containing the gene by using well-known gene manipulation techniques.

Polynucleotides according to the present invention can be prepared based on the sequence information concerning the specific example provided by the present invention, such as the polynucleotide shown by the nucleotide sequence set forth in SEQ ID NO: 1 in the sequence listing. The production of the polynucleotide can be carried out easily by using gene manipulation techniques that are well known per se (Sambrook et al., Eds., “Molecular Cloning, A Laboratory Manual, 2^{nd} Edition”, 1989, Cold Spring Harbor Laboratory: Muramatsu Masami., Ed., “Labomanual Genetic Engineering”, 1988, Maruzen Co., Ltd.).

Specifically, the polynucleotide of the present invention can be acquired by preparing a cDNA library in accordance with an ordinary method, from a suitable source in which expression of the polynucleotide was found, and then selecting a desired clone from the cDNA library. As a cDNA source, various kinds of cells and tissues in which expression of the present polynucleotide was found, or cultured cells derived from these cells and tissues, for example, cells derived from human spleen tissue, or the like, can be used. Isolation of total RNA from these sources, isolation and purification of mRNA, acquisition of cDNA, the cloning thereof, and the like, can each be performed in accordance with an ordinary method. It is also possible to use a cDNA library that was constructed from commercially available polyA⁺ RNA derived from human spleen tissue. A method for selecting a desired clone from a cDNA library is not particularly limited, and any methods generally used can be employed. For example, selection of a desired clone can be performed by using a probe or primer capable of selectively hybridizing to the present polynucleotide. Specifically, a plaque hybridization method, colony hybridization method, or the like, which uses a probe capable of selectively hybridizing to the present polynucleotide, or a combination of these methods, can be employed. As a probe, a polynucleotide chemically synthesized based on the sequence information of the present polynucleotide, and the like, can generally be used. The present polynucleotide or a polynucleotide shown by the partial nucleotide sequence of the present polynucleotide, is suitable for use as a probe, as well. Furthermore, a sense primer and an anti-sense primer, which were designed based on the sequence information of the present polynucleotide, can also be used as such a probe.

Selection of a desired clone from a cDNA library can be performed, for example, by detecting the expression of the protein in each clone, utilizing a known protein expression system, and further determining a biological function of the protein as an indicator. Any known expression system can be used as a protein expression system. For example, a cell free protein expression system can be conveniently used for determining the expression of protein (Madin, K. et al., Proceedings of The National Academy of Sciences of The United States of America, 2000, Vol. 97, p. 559-564).

A function of the proteins encoded by the polynucleotides according to the present invention, or a function of the proteins according to the present invention can be, for example, preferably a GEF activity for Rho family proteins, and more preferably a function of binding to Rho family proteins to exhibit a GEF activity. In other words, it can be, for example, preferably a function of activating Rho family proteins, and more preferably a function of binding to Rho family proteins to activate the Rho family proteins.

The polynucleotide according to the present invention can be also prepared preferably by using a DNA/RNA amplification method. For example, a polymerase chain reaction can be used (hereinafter, may be abbreviated as PCR: Ulmer, K. M. Science, 1983, Vol. 219, p. 666-671; Ehrlich, H. A., Ed., PCR Technology. Principles and Applications for DNA Amplification, 1989, Stockton/Press; and Saiki, R. K. et al., Science, 1985, Vol. 230, p. 1350-1354). When the full-length cDNA is difficult to obtain from a cDNA library, a RACE method (Jikken Igaku (Experimental Medicine), 1994, Vol. 12, No. 6, p. 615-618), particularly the 5′-RACE method (Frohman, M. A. et al., Proceedings of The National Academy of Sciences of The United States of America, 1988, Vol. 85, No. 23, p. 8998-9002), or the like, can be suitably employed. Primers to be used for PCR can be suitably designed based on the nucleotide sequence information of the polynucleotide, and can be obtained by synthesis in accordance with any conventional method. Isolation and purification of amplified DNA/RNA fragments can be carried out according to any conventional method, such as gel electrophoresis, or the like.

A determination of the nucleotide sequence of DNA thus obtained can be carried out by any conventional method, such as the dideoxy method (Sanger, F. et al., Proceedings of The National Academy of Sciences of The United States of America, 1977, Vol. 74, p. 5463-5467), and the Maxam-Gilbert method (Maxam, A. M. et al., Methods in Enzymology, 1980, Vol. 65, p. 499-560), or by simply using a commercially available sequencing kit, or the like.

The polynucleotides according to the present invention are not limited to the aforementioned polynucleotides. They can include polynucleotides having a sequence homology with the aforementioned polynucleotides, which preferably encode proteins that bind to Rho family proteins, and more preferably encode proteins that bind to Rho family proteins to exhibit a GEF activity, or polynucleotides shown by the complementary nucleotide sequences of the polynucleotides. A suitable sequence homology with the entire base sequence is normally 50% or more, preferably at least 70%. It is more preferably 70% or more, even more preferably 80% or more, and still more preferably 90% or more. Further, polynucleotides having a DH/PH domain coding region are still more preferable for such polynucleotides. A suitable sequence homology with the DH/PH domain coding region is preferably at least 70%. It is more preferably 70% or more, even more preferably 80% or more, and still more preferably 90% or more. In addition, it is still more preferable that the DH/PH domain has its specific function such as a function of binding to Rho family proteins to exhibit a GEF activity.

The polynucleotides according to the present invention include polynucleotides shown by a nucleotide sequence with a mutation, such as deletion, substitution, addition or insertion, of one or more nucleotides in the nucleotide sequences of the aforementioned DNA, or polynucleotides shown by complementary nucleotide sequences. The number of mutated nucleotides is, for example, from 1 to 100, preferably from 1 to 30, more preferably from 1 to 20, even more preferably from 1 to 10, and still more preferably from 1 to several in number. The extent of mutation, the position of a mutation, and the like, are not particularly limited, as long as the polynucleotides with mutations preferably encode proteins that bind to Rho family proteins, and more preferably encode proteins that bind to Rho family proteins to exhibit a GEF activity. A polynucleotide that encodes a protein having a DH/PH domain is still more preferable. The polynucleotides with mutations may be natural polynucleotides, or may be mutated polynucleotides. Further, they also may be polynucleotides prepared by introducing a mutation into a natural gene. Techniques for introducing a mutation are known in the art. For example, site-directed mutagenesis, genetic homologous recombination, primer extension, PCR, and the like, can be used independently or in suitable combinations. Specifically, for example, a method described in publications (Sambrook et al., Eds., “Molecular Cloning, A Laboratory Manual, 2^{nd} Edition”, 1989, Cold Spring Harbor Laboratory; and Muramatsu Masami., Ed., “Labomanual Genetic Engineering”, 1988, Maruzen Co., Ltd.), or a modified method thereof, can be used for conducting the introduction of a mutation. In addition, Ulmer's techniques (Ulmer, K. M. Science, 1983, Vol. 219, p. 666-671) can also be utilized.

The polynucleotides of the present invention can also be polynucleotides that hybridize to the aforementioned polynucleotides under stringent conditions. A hybridization condition can be found, for example, in a method described in publications (Sambrook et al., Eds., “Molecular Cloning, A Laboratory Manual, 2^{nd} Edition”, 1989, Cold Spring Harbor Laboratory), or the like. More specifically, the phrase “under stringent conditions” refers to, for example, a condition of heating at 42° C. in a solution containing 6×SSC, 0.5% SDS and 50% formamide, and then washing at 68° C. in a solution containing 0.1×SSC and 0.5% SDS. Such polynucleotides are not required to have complementary sequences with the present polynucleotides, as long as they hybridize to the present polynucleotides. It is desirable that a polynucleotide preferably encodes a protein having a DH/PH domain. It is more desirable that the encoded protein is a protein that binds to Rho family proteins, and more preferable a protein that binds to Rho family proteins to exhibit a GEF activity.

The polynucleotides of the present invention also include oligonucleotides shown by a partial nucleotide sequence of a given region of the aforementioned polynucleotides. The minimum unit of such an oligonucleotide consists of consecutive nucleotides within the region, preferably of 5 or more, more preferable of 10 or more, and even more preferably of 20 or more consecutive nucleotides. Specifically, an oligonucleotide shown by the nucleotide sequence set forth in SEQ ID NO: 5, or SEQ ID NO: 6, can be preferably exemplified. These oligonucleotides can be used, for example, as primers for amplifying the present genes or the present gene figments, and as probes for detecting the present genes or their transcription products thereof. These oligonucleotides can be prepared by designing a desired sequence based on the nucleotide sequence information of the present polynucleotides, and then synthesizing them using a well-known chemical synthesis method. An automated DNA/RNA synthesizer can be conveniently used for preparing the oligonucleotides.

The polynucleotides of the present invention are preferably human derived polynucleotides. However, the present invention includes polynucleotides derived from mammals, for example, a polynucleotide derive from mouse, horse, sheep, cow, dog, monkey, cat, bear, rat, rabbit or the like, as long as the polynucleotide is a polynucleotide that has a sequence homology with the present polynucleotides, and that encodes a protein that binds to Rho family proteins to exhibit a GEF activity. A suitable sequence homology with the entire base sequence is normally 50% or more, preferably at least 70%. It is more preferably 70% or more, even more preferably 80% or more, and still more preferably 90% or more. Further, polynucleotides having a DH/PH domain coding region are more preferable for such polynucleotides. A suitable sequence homology with the DH/PH domain coding region is preferably at least 70%. It is more preferably 70% or more, even more preferably 80% or more, and still more preferably 90% or more.

The polynucleotides of the present invention also may have a desired gene at the 5′-terminal side or the 3′-terminal side thereof, as long as the expression of the polynucleotides or the function of proteins encoded by the polynucleotides is not inhibited. A gene capable of being added to the present polynucleotide can be specifically exemplified by the genes of enzymes, such as glutathione S-transferase (GST), β-galactosidase, horseradish peroxidase (HRP) or alkaline phosphatase (ALP), or of tag peptides such as His-tag, Myc-tag, HA-tag, FLAG-tag or Xpress-tag. One of these genes, or multiple kinds of these genes in combination, can be added to the present polynucleotides. Addition of these genes can be performed by using conventional gene manipulation techniques, and is useful to facilitate detection of a gene or mRNA.

(Vector)

Another aspect of the present invention relates to recombinant vectors containing a polynucleotide of the present invention. The recombinant vectors can be prepared by inserting the present polynucleotides into a suitable vector DNA.

The vector DNA is not particularly limited, as long as it can be replicated within a host, and can be suitably selected in accordance with the kind of host and purpose of use. The vector DNA may be vector DNA obtained by extracting natural DNA, or may be vector DNA lacking a part of DNA other than a segment necessary for replication. Typical vector DNAs include, for example, a vector DNA derived from a plasmid, a bacteriophage or a virus. A plasmid DNA can be exemplified by a plasmid derived from Escherichia coli, a plasmid derived from Bacillus subtilis, or a plasmid derived from yeast. A bacteriophage DNA can be exemplified by a λ phage. Vector DNA derived from a virus can be exemplified by a vector derived from an animal virus, such as a retrovirus, vaccinia virus, adenovirus, papovavirus, SV 40, fowlpox virus, and pseudorabies virus, or a vector derived from an insect virus such as baculovirus. Further, vector DNA derived from a transposon, an insertion element, a yeast chromosome element, or the like, may be exemplified. Alternatively, a vector DNA prepared by combining two or more of these, for example, a vector DNA (cosmid, phagemid or the like) prepared by combining genetic elements of a plasmid and a bacteriophage, may be exemplified.

Any vector DNA can be used in accordance with the desired purpose, for example, an expression vector, cloning vector or the like. The recombinant expression vector containing the polynucleotide of the present invention is useful for the production of proteins encoded by the present polynucleotides.

It is necessary for the polynucleotide of the present invention to be incorporated into vector DNA in such a way as to allow the function of the polynucleotide to appear. The vector DNA contains at least one of the present polynucleotides and a promoter, as construction elements. In addition to these elements, as desired, a genetic sequence that encodes information relating to replication and control, may be incorporated in combination into the vector DNA, by using a well-known method. Such a genetic sequence can be exemplified by a ribosome binding sequence, terminator, signal sequence, cis element such as an enhancer, splicing signal, and a selective marker such as dihydrofolate reductase gene, ampicillin-resistant gene and neomycin-resistant gene. The vector DNA may contain one or more kinds of genetic sequences selected from the aforementioned members.

As a method of incorporating the polynucleotide, according to the present invention, into a vector DNA, any known method can be employed. For example, a method may be used which comprises cleaving a gene containing the present polynucleotide at specific sites, by treating it with suitable restriction enzymes, and then mixing it with a similarly treated vector DNA, for ligation using a ligase. Alternatively, a desired recombinant vector may be prepared by using a method that comprises ligating one of the present polynucleotides with a suitable linker, and then inserting it into the multi-cloning site of a vector, suitable for the desired purpose.

(Transformant)

Further aspect of the present invention relates to transformants obtained by transforming a host with the recombinant vectors according to the present invention. A transformant, prepared by introducing a recombinant expression vector that contains a polynucleotide according to the present invention, is useful for producing a protein encoded by the present polynucleotides. The present transformants may further incorporate one or more kinds of vector DNAs, each containing a desired gene other than the present polynucleotides. A vector DNA that contains a desired gene other than a present polynucleotide, can be exemplified by vector DNA that contains a gene encoding a Rho family protein, such as, RhoA, or the like. A transformant prepared by transfecting with both of the expression vectors, one of which contains a present polynucleotide and the other contains a gene encoding a Rho family protein, may be used for a method of identifying a compound that inhibits or enhances a GEF activity of a protein encoded by a present polynucleotide for Rho family proteins. That is to say, the transformant may be used for a method of identifying a compound that inhibits or enhances the activation of Rho family proteins. Such a transformant can be preferably exemplified by a transformant prepared by transfecting with both recombinant vectors, one of which contains the present polynucleotide and the other contains a gene encoding RhoA.

Any suitable prokaryotes and eukaryotes can be employed as a host. Examples of suitable prokaryotes include bacteria belonging to the Escherichia genus, such as, Escherichia coli, bacteria belonging to the Bacillus genus, such as, Bacillus subtilis, bacteria belonging to the Pseudomonas genus, such as, Pseudomonas putida, and bacteria belonging to the Rhizobium genus, such as, Rhizobium meliloti. Examples of suitable eukaryotes include yeasts, and animal cells such as insect cells and mammalian cells. Yeasts can be exemplified by Saccharomyces cerevisiae and Schizosaccharomyces pombe. Insect cells can be exemplified by Sf9 cells and Sf2 cells. Mammalian cells can be exemplified by monkey kidney-derived cells, such as COS cells, Vero cells, Chinese hamster ovary cells (CHO cell), mouse L cells, rat GH3 cells, human FL cells, and 293EBNA cells. It is preferable to use mammalian cells, and more preferable to use 293EBNA cells.

Transformation of a host cell with a recombinant vector can be carried out by utilizing known methods. For example, a standard method described in publications (Sambrook et al., Eds., “Molecular Cloning, A Laboratory Manual, 2^{nd} Edition”, 1989, Cold Spring Harbor Laboratory) may be utilized. When gene stability is a consideration, it is preferable to use a method that integrates the gene onto a chromosome. Meanwhile, it is convenient to use an autonomous replication system that utilizes an extranuclear gene. Specifically, calcium phosphate transfection, DEAE-dextran mediated transfection, microinjection, cationic lipid-mediated transfection, electroporation, transduction, scrape loading, ballistic introduction, infection, and the like, may be mentioned.

When employing a prokaryote as a host, it is preferable to use a recombinant vector which is capable of autonomous replication within the bacterium, and is also composed of a promoter, a ribosomal binding sequence, the polynucleotide of the present invention, and a transcription termination sequence. It may also contain a gene that regulates the promoter. When employing bacteria as a host, any promoter may be used, as long as it can lead to expression in bacteria, such as Escherichia coli. For example, a promoter derived from Escherichia coli or a phage can be used, such as a trp promoter, lac promoter, PL promoter or PR promoter. An artificially designed and modified promoter such as a tac promoter may also be used. A method of introducing a recombinant vector into bacteria is not particularly limited, and any methods that introduce DNA into bacteria can be employed. Preferable examples of such a method include using calcium ions, electroporation, or the like.

When employing a mammalian cell as a host, it is preferable to use the recombinant vector which is capable of autonomous replication within the cell, and is also composed of a promoter, RNA splice site, a polynucleotide of the present invention, polyadenylated site and a transcription termination sequence. As desired, it may also contain an origin of replication. A SRα promoter, SV 40 promoter, LTR promoter, CMV promoter, and the like, can be used as a promoter. An early gene promoter of cytomegalovirus, and the like, may be used, as well. As a method of introducing the recombinant vector into a mammalian cell, preferably, for example, electroporation, the calcium phosphate technique, lipofection, or the like, may be used. A most preferable method to be used may be lipofection.

When using yeast as a host, the promoter is not particularly limited, as long as it can lead to expression in yeast. Examples of such a promoter include the gal1 promoter, gal10 promoter, heat shock protein promoter, MFα1 promoter, PHO5 promoter, PGK promoter, GAP promoter, ADH promoter, and AOX1 promoter. A method of introducing a recombinant vector into yeast is not particularly limited, as long as it is a method that introduces the DNA into the yeast. Preferable examples of such a method include electroporation, a spheroplast method, a lithium acetate method or the like.

When using an insect cell as a host, it is preferable to use a calcium phosphate technique, lipofection, or electroporation for a method of introducing a recombinant vector.

(Protein)

A further aspect of the present invention relates to proteins encoded by a polynucleotide according to the present invention.

The protein according to the present invention can be, for example, a protein encoded by the polynucleotide shown by the nucleotide sequence set forth in SEQ ID NO: 1. More specifically, the protein can be a protein shown by the amino acid sequence set forth in SEQ ID NO: 2. The protein shown by the amino acid sequence set forth in SEQ ID NO: 2 has a DH domain composed of the amino acid residues from the 1178^{th} valine (Val) to the 1357^{th} asparagine (Asn). The protein has a PH domain composed of the amino acid residues from the 1391^{st} leucine (Leu) to the 1502^{nd} glutamic acid (Glu). Thus, the protein has a DH/PH domain composed of the amino acid residues from the 1178^{th} valine (Val) to the 1502^{nd} glutaric acid (Glu).

A protein according to the present invention may be preferably a protein that binds to Rho family proteins. The protein encoded by the polynucleotide shown by the nucleotide sequence set forth in SEQ ID NO: 1, was bound to a Rho family protein, RhoA. Specifically, in an animal cell into which the polynucleotide shown by the nucleotide sequence set forth in SEQ ID NO: 1 was co-expressed with a gene encoding RhoA, the binding of a protein encoded by the polynucleotide to RhoA was detected (See Example 3). The polynucleotide shown by the nucleotide sequence set forth in SEQ ID NO: 1, encodes the protein shown by the amino acid sequence set forth in SEQ ID NO: 2. Therefore, it can be considered that the protein shown by the amino acid sequence set forth in SEQ ID NO: 2, was bound to RhoA. On the other hand, in an animal cell into which the polynucleotide shown by the nucleotide sequence set forth in SEQ ID NO: 3 was co-expressed with a gene encoding RhoA, no binding was detected between a protein encoded by the polynucleotide and RhoA (See Example 3). The polynucleotide shown by the nucleotide sequence set forth in SEQ ID NO: 3 is a polynucleotide shown by the nucleotides from the 175^{th} nucleotide to the 3693^{rd} nucleotide of the nucleotide sequence set forth in SEQ ID NO: 1. The polynucleotide shown by the nucleotide sequence set forth in SEQ ID NO: 3, encodes a protein shown by the amino acid sequence set forth in SEQ ID NO: 4. The protein shown by the amino acid sequence set forth in SEQ ID NO: 4 lacks a region shown by the amino acid resides from the 1174^{th} amino acid to the 1597^{th} amino acid in the amino acid sequence set forth in SEQ ID NO: 2. The region contains a DH/PH domain. Thus, the protein shown by the amino acid sequence set forth in SEQ ID NO: 4 lacks a DH/PH domain. And, the protein shown by the amino acid sequence set forth in SEQ ID NO: 4 lacking a DH/PH domain did not bind to RhoA. These findings revealed that a DH/PH domain participated in the binding of the protein shown by the amino acid sequence set forth in SEQ ID NO: 2 to RhoA.

A protein according to the present invention has a DH/PH domain in the structure and binds to Rho family proteins. A DH/PH domain is important for the binding of Rho-GEF to Rho family proteins, and further for a GEF activity of Rho-GEF. Therefore, the inventors believe that the protein binds to Rho family proteins to exhibit a GEF activity for the Rho family proteins. More specifically, the inventors believe that the protein shown by the amino acid sequence set forth in SEQ ID NO: 2 binds to a Rho family protein, RhoA, and exhibits a GEF activity. In other words, the inventors believe that the present protein binds to RhoA and activates it.

The protein according to the present invention is not limited to a protein as exemplified above, and any protein is included in the scope of the present invention, as long as it is encoded by a polynucleotide according to the present invention. Preferably, a protein that is encoded by a present polynucleotide and that binds to Rho family proteins may be included. More preferably, a protein that is encoded by a present polynucleotide and that binds to Rho family proteins to exhibit a GEF activity may be included. Such a protein can be exemplified by a protein that is encoded by a polynucleotide shown by a nucleotide sequence having a homology of at least 70% with the nucleotide sequence of any one of the polynucleotides selected from the group consisting of the following: a polynucleotide shown by the nucleotide sequence set forth in SEQ ID NO: 1, or its complementary nucleotide sequence; and a polynucleotide encoding the protein shown by the amino acid sequence set forth in SEQ ID NO: 2, or a polynucleotide shown by the complementary nucleotide sequence of the polynucleotide, where the protein has a GEF activity for a Rho family protein. Further, the present protein also includes a protein that is encoded by a polynucleotide shown by a nucleotide sequence with a mutation or a induced mutation, such as deletion, substitution, addition or the like, of one or more nucleotides in the nucleotide sequence of any one of polynucleotides selected from the aforementioned polynucleotide group, where the protein is encoded by a polynucleotide encoding a protein that has a GEF activity for a Rho family protein. Furthermore, the present protein may also be exemplified by a protein encoded by a polynucleotide that hybridizes to any one of the polynucleotides selected from the aforementioned polynucleotide group, under stringent conditions, and encodes a protein that preferably bind to a Rho family protein, more preferably bind to a Rho family protein to show a GEF activity for a Rho family protein.

A protein according to the present invention can be more specifically, for example, a protein that has a sequence homology with the protein shown by the amino acid sequence set forth in SEQ ID NO: 2, and that binds to a Rho family protein. More preferably, it may be a protein that has a sequence homology with the protein shown by the amino acid sequence set forth in SEQ ID NO: 2, and that binds to a Rho family protein to exhibit a GEF activity. A suitable sequence homology with the entire amino acid sequence is normally 50% or more, preferably at least 70%. It is more preferably 70% or more, even more preferably 80% or more, and still more preferably 90% or more. Further, a polypeptide having a DH/PH domain is still more preferable. A suitable sequence homology with the DH/PH domain is preferably at least 70%. It is more preferably 70% or more, even more preferably 80% or more, and still more preferably 90% or more. In addition, it is still more preferable that the DH/PH domain has its specific function. Further, a protein of the present invention includes a protein that is shown by an amino acid sequence with a mutation, such as deletion, substitution, addition, or the like, of one or more amino acids in the amino acid sequence set forth in SEQ ID NO: 2, and that binds to a Rho family protein. The mutation of amino acids is, for example 1 to 100, preferably 1 to 30, more preferably 1 to 20, further preferably 1 to 10, and still more preferably 1 to several in number. More preferably, it may be a protein that that has a sequence homology with the protein shown by the amino acid sequence set forth in SEQ ID NO: 2, and that binds to a Rho family protein to exhibit a GEF activity. The extent of mutation, the position of a mutation, and the like, of the amino acid are not particularly limited, as long as a protein with a mutation binds to a Rho family protein, preferably bind to a Rho family protein to exhibit a GEF activity. Still more preferably, it may be a protein having a DH/PH domain. Such a protein with a mutation may be a protein generated in nature, for example, due to mutation or post translational modification. Further, it also may be a protein prepared by introducing a mutation into a natural gene. Techniques for introducing a mutation are known in the art. For example, known gene manipulation techniques can be used for preparation. When introducing a mutation, in view of avoiding a change in the fundamental properties (such as physical properties, function, physiological activity, and immunological activity) of the protein, mutual substitution among homologous amino acids (polar amino acids, non-polar amino acids, hydrophobic amino acids, hydrophilic amino acids, positively-charged amino acids, negatively-charged amino acids and aromatic amino acids or the like) may be readily conceived.

A protein according to the present invention further includes a protein shown by a partial sequence of the aforementioned protein. For example, a protein that is shown by a partial sequence of the protein shown by the amino acid sequence set forth in SEQ ID NO: 2, is also included in the scope of the present invention. The minimum unit of such a protein consists of consecutive amino acids, preferably 5 or more, more preferably 8 or more, even more preferably 12 or more, or 15 or more.

A protein of the present invention is preferably a human derived protein. However, the present invention includes proteins derived from mammals, for example, proteins derived from a mouse, horse, sheep, cow, dog, monkey, cat, bear, rat, rabbit, or the like, as long as the protein is a protein that has a sequence homology with the present proteins and preferably binds to a Rho family protein, and more preferably binds to a Rho family protein to exhibit a GEF activity. A suitable sequence homology with the entire amino acid sequence is normally 50% or more, preferably at least 70%. It is more preferably 70% or more, even more preferably 80% or more, and still more preferably 90% or more. Further, a protein having a DH/PH domain is more preferable for such a protein. A suitable sequence homology with the DH/PH domain is preferably at least 70%. It is more preferably 70% or more, even more preferably 80% or more, and still more preferably 90% or more.

A protein of the present invention may be a protein prepared from a cell in which a gene encoding the present protein is expressed by means of a gene manipulation technique, or from any suitable biological sample. A protein also may be a product of a cell-free synthesis system, or a chemical synthesis product. These can be subsequently further purified for use. In addition, the present protein may be a protein being expressed in a cell that contains a gene encoding the present protein. The cell may be a transformant prepared by transfecting a vector that contains a gene encoding the present protein.

A protein of the present invention can be modified to the extent that no significant functional change is involved, such as amidation of its constituent amino groups, carboxyl groups, or the like. A present protein can also be labeled with the other protein, or the like, that is added to the N-terminal or C-terminal, directly or indirectly, via a linker peptide, or the like, or by means of gene manipulation techniques, or the like. Labeling is preferably conducted in a way not to inhibit the fundamental properties of the present protein. A substance used for labeling (a labeling substance) can be exemplified by enzymes such as GST, β-Gal, HRP, ALP, or the like, tag peptides such as His-tag, Myc-tag, HA-tag, FLAG-tag, Xpress-tag or the like, fluorescent substances, such as fluorescein isothiocyanate, phycoerythrin or the like, a maltose binding protein, an immunogloblin Fc-fragment, biotin, or the like. However, it is not limited to these specific examples. Labeling can also be carried out using a radioactive isotope. One or more kinds of labeling substances in combination can be added to the present protein. These labeling substances allow the detection and/or purification of the present protein to become easier, by measuring the substance itself, or the function thereof. In addition, these substances allow, for example, the detection of the binding of the present protein to the other protein, and the measurement of the function of the present protein.

(Method of Producing a Protein)

A further aspect of the present invention relates to a method of producing a protein according to the present invention. The present protein can be prepared, for example, by standard gene manipulation techniques (refer to Sambrook et al., Eds., “Molecular Cloning, A Laboratory Manual, 2^{nd} Edition”, 1989, Cold Spring Harbor Laboratory; Muramatsu Masami., Ed., “Labomanual Genetic Engineering”, 1988, Maruzen Co., Ltd.; Ulmer, K. M. Science, 1983, Vol. 219, p. 666-671; Ehrlich, H. A., Ed., PCR Technology. Principles and Applications for DNA Amplification, 1989, Stockton Press and the like) based on the nucleotide sequence information of a gene encoding the present protein. For example, at first, a cDNA library may be prepared from various kinds of cells or tissues in which the expression of the present polynucleotide was found, or cultured cells derived from these cells and tissues, in accordance with conventional methods. Then, the polynucleotide encoding the present protein may be amplified from the cDNA library, by using a primer that selectively hybridizes to the gene encoding the protein. The amplified polynucleotide may be subjected to the expression induction by using known gene manipulation techniques to produce the present protein.

Specifically, for example, the present proteins can be produced by culturing the transformants according to the present invention, and then collecting the present proteins from the culture product obtained. The transformant can be cultured according to known culture conditions and culture methods that are suitable for a host used for the preparation of the transformant Cultivation can be carried out by employing an indicator, such as the present proteins themselves that are expressed by the transformant, or a function thereof. Alternatively, cultivation may be carried out by employing such an indicator as the present proteins themselves produced in a host or outside a host, or the amount of the protein. Otherwise, subculturing or batch culturing may be carried out by employing such an indicator as the amount of transformant in the culture medium.

When the proteins according to the present invention are expressed in a transformant, or on its cell membrane, the proteins may be extracted from the disrupted transformant Further, when the present proteins are secreted outside the transformant, the cultured medium can be used as is, or the cultured medium can be used after removing the transformant by centrifugation or the like. As desired, the present protein can be isolated and/or purified from a cultured medium of the transformant or from the transformant.

Purification and/or isolation of the protein according to the present invention can be carried out by various isolation methods that utilize the physical properties or chemical properties of the present protein. Specific examples of the isolation methods may include ammonium sulfate precipitation, ultrafiltration, gel chromatography, ion-exchange chromatography, affinity chromatography, high performance liquid chromatography, and dialysis. These methods may be used in suitable combinations. It is preferable to employ an affinity chromatography that utilizes a column bound with a specific antibody specific to the present protein. The specific antibody to the present protein can be prepared based on the amino acid information of the protein by a well known method of producing an antibody. The purification and/or isolation may be carried out by employing an indicator to obtain the present protein, such as a function of the present protein.

The proteins of the present invention can also be produced according to conventional chemical synthesis methods. As a chemical synthesis method of a protein, for example, methods described in publications (“peptide synthesis”, Maruzen Co., Ltd., 1975; and “peptide synthesis”, Interscience, New York, 1996) may be used. However, it is not limited to these methods, and any known methods can be used. Specifically, solid phase synthesis, solution phase synthesis, and the like, are known as chemical synthesis methods for proteins, and any of these methods can be used. These kinds of protein synthesis methods more specifically include a so-called stepwise elongation method that sequentially binds each amino acid, one at a time, to elongate a chain based on amino acid sequence information, and a fragment condensation method that previously synthesizes fragments consisting of several amino acids, and subsequently subjects the respective fragments to a coupling reaction. The present proteins can be synthesized by either of these methods. A condensation method used for the aforementioned protein synthesis methods can also be carried out according to conventional methods. Examples of condensation methods include an azide method, mixed anhydride method, DCC method, active ester method, oxidation-reduction method, DPPA (diphenylphosphoryl azide) method, DCC+additive (1-hydroxybenzotriazole, N-hydroxysuccinamide, N-hydroxy-5-norbomane-2,3-dicarboxylmide, and the like) method, and Woodward's method. The present protein obtained by chemical synthesis can be suitably purified in accordance with various kinds of conventional purification methods as described above.

Proteins shown by partial sequences of the proteins according to the present invention can also be obtained by cleaving the proteins according to the present invention, by a suitable peptidase.

(Antibody)

A further aspect of the present invention relates to antibodies recognizing the proteins according to the present invention. The antibodies can be prepared using the present proteins as antigens. The antigens to be used may be the present proteins or fragments thereof. The antigen consist of amino acids of at least eight, preferably at least ten, more preferably at least twelve, and even more preferably fifteen or more amino acids. In order to prepare specific antibodies to the present proteins, it is preferable to use a region comprising a characteristic amino acid sequence of the present proteins. The amino acid sequence of this region is not necessarily required to be identical to an amino acid sequence of the present proteins or fragments thereof. An amino acid sequence of a site that is exposed outward on a tertiary structure thereof, may be preferable for such an amino acid sequence. Even if the amino acid sequence of the exposure site is not continuous on the prima structure, it is sufficient for the amino acid sequence to be continuous with respect to the exposure site. The antibodies are not particularly limited, and can be any antibody, as long as it can specifically recognize the present proteins. The phrase “specifically recognize the present proteins” means to bind to the present proteins, but not bind to or weakly bind to other proteins than the present proteins. The presence or absence of the recognition can be determined by known antigen-antibody binding reactions.

The antibodies can be produced by utilizing known antibody producing methods. For example, the antibodies can be obtained by administering to an animal an antigen alone, or an antigen bound to a carrier, with or without an adjuvant, and thereby inducing immunity, such as a humoral response, and/or a cellular response. Any known carrier can be used, as long as it does not exhibit an adverse action against the host, and is capable of enhancing the antigenicity of the antigen. Specifically, examples of the carrier include cellulose, polymeric amino acids, albumin, and keyhole limpet hemocyanin. Examples of the adjuvant include Freund's complete adjuvant (FCA), Freund's incomplete adjuvant (FIA), Ribi (MPL), Ribi (TDM), Ribi (MPL+TDM), Bordetella pertussis vaccine, muramyldipeptide (MDP), aluminium adjuvant (ALUM), and combinations of these. Mouse, rat, rabbit, goat, horse, or the like, can be preferably used as animals for immunization.

A polyclonal antibody can be acquired from the serum of an animal that was administered with an antigen by using any known method for recovering an antibody. Immunoaffinity chromatography may be employed as a preferable example of the method for recovering an antibody.

A monoclonal antibody can be produced utilizing a hybridoma that is prepared by collecting an antibody-producing cell, (for example, lymphocytes derived from spleen or lymph nodes) from an animal that was administered with an antigen, and then fusing it with a well known immortalized cell (for example, myeloma strain such as P3-X63-Ag8 line). For example, the antibody-producing cell is fused with an immortalized cell by any known method to prepare a hybridoma which is subsequently subjected to cloning. The various cloned hybridomas are used to screen for a hybridoma that produces an antibody specifically recognizing a protein of the present invention. The antibody can be then recovered from a culture solution of that hybridoma.

A polyclonal antibody or monoclonal antibody, which is capable of recognizing or binding to a protein of the present invention, can be utilized as an antibody for purification of the present protein, reagent, labeling marker or the like. In particular, an antibody that inhibits the function of the present protein can be used for regulating the function of the present protein, and is useful for elucidating, preventing, improving, and/or treating various kinds of diseases due to an abnormality of a present protein, in amount and/or function.

(Method of Identifying a Compound)

A still further aspect of the present invention relates to methods of identifying compounds that inhibit or enhance the function of the proteins according to the present invention, or compounds that inhibit the expression of the polynucleotides according to the present invention. Since it was found that the expression of the present polynucleotide is high in tonsil tumors such as a tonsil squamous cell carcinoma, and in bronchial tumors such as a bronchial adenoid cystic carcinoma, the present inventors believe that prevention and/or treatment of these diseases can be conducted by inhibiting the function of the present proteins and/or inhibiting the expression of the present polynucleotides. Therefore, an identification method according to the present invention may be preferably a method of identifying a compound that inhibits the function of the present proteins, or compounds that inhibit the expression of the present polynucleotides.

The identification methods according to the present invention can be carried out using at least one member selected from the proteins, the polynucleotides, the recombinant vectors, the transformants, and the antibodies which are provided in the present invention, by employing any known pharmaceutical screening system. The present identification methods include any methods that are carried out in vitro or in vivo. The present identification methods allow screening for antagonists by drug design based on the structure of the present proteins, screening for an inhibitor of the expression at the gene level by utilizing a protein synthesis system, or screening for a substance recognized by an antibody by utilizing the antibody, or the like.

A method of identifying a compound that inhibits or enhances the function of the proteins according to the present invention can be carried out using an experimental system capable of measuring the function of the present protein, which comprises allowing the present protein coexist with a compound to be tested (test compound), under conditions allowing the interaction of the present protein with the test compound, and measuring the function of the present protein, subsequently, comparing the function of the present protein in the presence of the test compound to the function of the present protein in the absence of the test compound, and finally detecting the presence, the absence, or the change of the function of the present protein, such as reduction, enhancement, disappearance, and appearance. Where the function of the present protein is reduced or eliminated in the presence of a test compound, in comparison to the function of the present protein in the absence of the test compound, it can be determined that the test compound inhibits the function of the present protein. Oppositely, where the function of the present protein is enhanced or appeared in the presence of a test compound, it can be determined that the test compound enhances the function of the present protein. The function of the proteins can be measured by direct detection of the function, or by introducing a signal as an indicator of the function into an experimental system and detecting the signal. Examples of a signal include enzymes such as GST, tag peptides such as His-tag, Myc-tag, HA-tag, FLAG-tag, or Xpress-tag, or a fluorescent protein. Any other labeling substance can be used, as long as it is used in a conventional method of identifying a compound.

The function of the proteins according to the present invention may be, for example, preferably a GEF activity for a Rho family protein. The present proteins have a DH/PH domain that is an active domain of Rho-GEF and is required for the domain for binding to a Rho family protein. Therefore, the present inventors believe that a compound that inhibits the function of the present proteins can be identified by employing an indicator, such as the binding of the present proteins to a Rho family protein.

An identification method employing such an indicator as the binding of the proteins according to the present invention to a Rho family protein, can be carried out, for example, by expressing the present proteins using gene manipulation techniques to obtain the proteins, and detecting the binding of the proteins to a Rho family protein in the presence or absence of a test compound. Specifically, for example, the present proteins can be subjected to a reaction in the presence or absence of a test compound, with a Rho family protein that is expressed as a GST-fusion protein by using gene manipulation techniques followed by binding to a glutathione-Sepharose. The identification of a compound that inhibits or enhances the binding of the present proteins to a Rho family protein can be achieved by measuring the present proteins that bind to the Rho family protein bound to a glutathione-Sepharose. Where the binding of both proteins is reduced or eliminated, in the presence of a test compound, in comparison to the binding of both proteins in the absence of the test compound, it can be determined that the test compound inhibits the binding of the present proteins to the Rho family proteins. Oppositely, where the binding of both proteins is increased in the presence of a test compound, it can be determined that the test compound enhances the binding of the present protein to the Rho family protein. The quantitative measurement of the present proteins can be carried out, for example, by using antibodies according to the present invention. An antibody labeled with a labeling substance such as enzymes (e.g. HRP or ALP), a radioactive isotope, a fluorescent substance, or biotin may be used. Alternatively, a labeled second antibody may be used. In the case of using the present proteins fused with a tag-peptide, the quantitative measurement thereof can be carried out using an antibody against the tag-peptide. Alternatively, the present proteins may be used after labeling directly with a labeling substance, such as the aforementioned enzyme, radioactive isotope, fluorescent substance, biotin, or the like. In such a case, the quantitative measurement can be carried out by measuring the labeling substance.

More specifically, an identification method employing such an indicator as the binding of the proteins according to the present invention to a Rho family protein, can be carried out using suitable cells in which a polynucleotide encoding the present protein are co-expressed with a polynucleotide encoding a Rho family protein, and using an experimental system which detects the binding of both proteins, by pull-down assay (see Example 3).

A well known two-hybrid method can be used for carrying out an identification method employing such an indicator as the binding of the proteins according to the present invention to a Rho family protein. For example, the method can be carried out wherein a plasmid for expressing a fusion protein of the present protein and a DNA binding protein, a plasmid for expressing a fusion protein of a Rho family protein and a transcription activating protein, and a plasmid containing a reporter gene that is linked to a suitable promoter gene, are introduced to a yeast, a eukaryotic cell, or the like. The identification of a compound that inhibits or enhances the binding of the present protein to a Rho family protein can be achieved by comparing the amount of expression of the reporter gene, in the presence of a test compound, to an amount of expression of the reporter gene in the absence of the test compound. In the case that the amount of expression of the reporter gene in the presence of the test compound is reduced or eliminated, compared to the amount of expression of the reporter gene in the absence of the test compound, it can be determined that the test compound inhibits the binding of the present protein to a Rho family protein. Oppositely, in the case that the amount of expression of the reporter gene in the presence of a test compound is increased, it can be determined that the test compound enhances the binding of the present protein to the Rho family protein. Any reporter genes that are used in a conventional reporter assay can be used herein. A reporter gene can be exemplified by a gene having an enzyme activity, such as, luciferase, β-Gal, chloramphenicol acetyl transferase, or the like. The expression of the reporter gene can be detected by determining the activity of the gene product, for example, an enzyme activity in the case of using the reporter gene exemplified in the above.

A surface plasmon resonance sensor, such as, the BIACORE system, or the like, can also be used in the identification method employing such an indicator as the binding of the proteins according to the present invention to a Rho family protein Alternatively, Scintillation proximity assay (SPA), or a method employing fluorescence resonance energy transfer (FRET), can also be used for carrying out the present identification method.

An identification method employing such an indicator as a GEF activity of the proteins according to the present invention for a Rho family protein, can be carried out, for example, by allowing a present protein to co-exist with a Rho family protein, which may be activated by the present protein, and measuring the amount of an activated Rho family protein in the presence or absence of a test compound. In the case that the amount of an activated Rho family protein is reduced in the presence of a test compound, compared to in the absence of the test compound, it can be determined that the test compound inhibits a GEF activity of the proteins according to the present invention for a Rho family protein. Oppositely, in the case that the amount of an activated Rho family protein is increased in the presence of a test compound, compared to in the absence of the test compound, it can be determined that the test compound enhances a GEF activity of the protein according to the present invention for a Rho family protein. An activated Rho family protein can be measured by using an antibody raised against the protein. For example, an activated Rho family protein can be measured using an effector molecule capable of binding to the activated Rho family protein, but not binding or weakly binding to the non-activated Rho family protein, by effector pull down assay.

Specifically, a Rho family protein activated by the proteins according to the present invention, can be quantitatively measured using suitable cells in which the polynucleotide encoding the protein according to the present invention was co-expressed with a polynucleotide encoding a Rho family protein. An activated Rho family protein can be quantitatively measured by utilizing a GST-fused effector molecule fragment containing a binding site of the protein, by effector pull down assay. Specifically, an activated Rho family protein is first subjected to a reaction with a GST-fused effector molecule fragment. After that, the effector molecule fragment is collected by using anti-GST-tag antibody. Then, the activated Rho family protein bound to the collected effector molecule fragment is quantitatively measured. An amount of the activated Rho family protein can be measured by electrophoresis and Western blotting. An effector molecule that binds to an activated Rho family protein is different depending on the type of a Rho family protein. Therefore, a suitable effector protein may be selected for use according to the kind of a Rho family protein used. For example, activated RhoA is known to bind to the effector molecule, Rhotekin. Therefore, Rhotekin may be a suitable effector molecule for use to identify a compound that inhibits or enhances the activation of RhoA.

An identification method employing such an indicator as a GEF activity of the proteins according to the present invention for a Rho family protein, can be also carried out by allowing the present proteins to co-exist with a Rho family protein, which is activated by the present proteins and is bound to radioactive isotope-labeled GDP, and with GTP, and subsequently measuring the amount of an activated Rho family protein in the presence or absence of a test compound. The activated Rho family protein can be quantitatively determined by measuring the reduced amount of the Rho family protein being bound to radioactive isotope-labeled GDP. Such a method can be carried out by referring a method described in a known literature (Journal of Biological Chemistry, 1996, Vol. 271, No. 44, p. 27374-27381).

The phrase “inhibiting a GEF activity for a Rho family protein” means to inhibit the conversion of a GDP-bound inactive form of Rho family protein to a GTP-bound active form of Rho family protein. More specifically, it means to reduce or to delete the amount of a GTP-bound active form of Rho family protein. The phrase “enhancing a GEF activity for a Rho family protein” means to enhance the conversion of a GDP-bound inactive form of Rho family protein to a GTP-bound active form of Rho family protein. More specifically, it means to increase the amount of a GTP-bound active form of Rho family protein.

A Rho family protein used in the identification method according to the present invention may be a protein lacking of a part thereof or a protein labeled with an aforementioned labeling substance, as long as the activation thereof by a protein according to the present invention is not affected. The labeling substance is exemplified in the above description.

An identification method employing such an indicator as the expression of a polynucleotide according to the present invention, can be carried out using an experimental system capable of measuring the expression of the present polynucleotide, which comprises allowing the present polynucleotide co-exist with a test compound under conditions allowing the interaction of the present polynucleotide with the test compound, and measuring the expression of the present polynucleotide. Subsequently, the expression of the present polynucleotide in the presence of the test compound can be compared to the expression of the present polynucleotide in the absence of the test compound, for detecting the presence, the absence, or the change of the expression of the present polynucleotide, such as reduction, enhancement, disappearance, and appearance. In the case that the expression of the present polynucleotide is reduced or disappeared in the presence of a test compound in comparison to the expression of the present polynucleotide in the absence of the test compound, it can be determined that the test compound inhibits the expression of the present polynucleotide. Oppositely, in the case that the expression of the present polynucleotide is increased in the presence of a test compound, it can be determined that the test compound enhances the expression of the present polynucleotide.

Specifically, for example, the identification method employing such an indicator as the expression of a polynucleotide according to the present invention, can be carried out using an experimental system using the transformant according to the present invention, for expressing the present polynucleotide, which comprises contacting the transformant with a test compound, and then measuring the expression of the present polynucleotide. The expression of the present polynucleotide can be measured easily by detecting the amount of expressed protein, or by detecting the function of the protein. Further, the expression of the present polynucleotide can be measured also by introducing, for example, a signal as an indicator of the expression into the experimental system, and detecting the signal. Examples of a signal include: enzymes such as GST; tag peptides such as His-tag, Myc-tag, HA-tag, FLAG-tag, or Xpress-tag; or a fluorescent substance. A method of detecting these signals is well known to those skilled in the art.

An identification method employing such an indicator as the expression of a polynucleotide according to the present invention, can also be carried out by, for example, preparing a vector that comprises a promoter region of a gene corresponding to the present polynucleotide and a reporter gene linked downstream of the promoter region instead of the present polynucleotide, and contacting a cell, e.g. a eukaryotic cell, which contains the vector, with the test compound, and then determining the presence or absence of, or a change in expression of the reporter gene. Any reporter genes that are used in a conventional reporter assay can be used herein. Specifically, a reporter gene can be exemplified by a gene having an enzyme activity, such as, luciferase, β-Gal, chloramphenicol acetyl transferase, or the like. The expression of the reporter gene can be detected by determining the activity of the gene product, for example, an enzyme activity in the case of using a reporter gene exemplified above.

(Compound)

A compound obtained by an identification method according to the present invention can be utilized as a candidate compound for an inhibitor of the expression of a polynucleotide according to the present invention, an inhibitor or an antagonist of the function of a protein according to the present invention. For example, the compound can be utilized as a candidate compound for an inhibitor of a GEF activity for a Rho family protein, namely, an inhibitor of an activation of a Rho family protein. A compound obtained by the present identification method can be utilized as a candidate compound for an agent for enhancing the expression of the polynucleotide according to the present invention, or an agent for enhancing the function of the protein according to the present invention. Since it was found that the expression of the present polynucleotide is high in tonsil tumors such as a tonsil squamous cell carcinoma, and in bronchial tumors such as an bronchial adenoid cystic carcinoma, the present inventors believe that prevention and/or treatment of the diseases can be conducted by inhibiting the function of the present protein and/or inhibiting the expression of the present polynucleotide. Therefore, a compound obtained by the identification method according to the present invention may be preferably a compound that inhibits the function of the present protein and/or a compound that inhibits the expression of the present polynucleotide. These candidate compounds can be prepared as a medicament by taking into consideration the balance between usefulness and toxicity. Such a medicament is useful for preventing and/or treating various kinds of symptoms due to an abnormality in the function of the present protein and/or an abnormality in the expression of the present polynucleotide. The compounds according to the present invention include compounds that are obtained by other methods than the present identification methods if they are capable of inhibiting the function of the present protein and/or the expression of the present polynucleotide, or are capable of enhancing the function of the present protein and/or the expression of the present polynucleotide.

(Pharmaceutical Composition)

A further aspect of the present invention relates to a medicament or a pharmaceutical composition which is based on inhibiting or antagonizing the function of a present protein and/or the expression of a present polynucleotide, or which is based on enhancing the function of the present protein and/or the expression of the present polynucleotide. The medicament or the pharmaceutical composition contains a protein, a polypeptide, a recombinant vector, a transformant, an antibody, or a compound, which is provided according to the present invention, as an active ingredient.

A medicament according to the present invention can be a medicament that contains an effective amount of at least one member selected from a protein, a polynucleotide, a recombinant vector, a transformant, an antibody, or the compound, which is provided according to the present invention, as an active ingredient. In general, it is preferable to prepare a pharmaceutical composition using one or more kinds of pharmaceutically acceptable carriers (pharmaceutical carriers).

An amount of the active ingredient contained in the pharmaceutical composition according to the present invention can be suitably selected from a wide range. In general, a suitable amount may fall within a range of approximately 0.00001 to 70 wt %, preferably approximately 0.0001 to 5 wt %.

A pharmaceutical carrier may be a diluent or excipient, which can be generally used in accordance with the form of use of the pharmaceutical composition, such as, a filler, an extender, a binder, a wetting agent, a disintegrator, and/or a lubricant. These can be suitably selected and used in accordance with the form of use of the pharmaceutical composition used.

The pharmaceutical carrier may be, for example, water, a pharmaceutically acceptable organic solvent, collagen, polyvinyl alcohol, polyvinylpyrrolidone, carboxyvinyl polymer, sodium alginate, soluble dextran, sodium carboxymethyl starch, pectin, xanthan gum, acacia gum, casein, gelatin, agar, glycerin, propylene glycol, polyethylene glycol, vaseline, paraffin, stearyl alcohol, stearic acid, human serum albumin, mannitol, sorbitol and lactose. One or a combination of two or more kinds of these carriers may be suitably selected, and used in accordance with the form of use of a pharmaceutical composition of the present invention.

As desired, various ingredients used in conventional protein preparations can be suitably used herein, such as, a stabilizer, a bacteriocide, a buffer agent, an isotonizing agent, a chelating agent, a pH adjuster, or a surfactant, for preparing the pharmaceutical composition.

As a stabilizer, the following may be used: human serum albumin, common L-amino acids, sugars, and cellulose derivatives. These can be used independently or in combination with a surfactant, and the like. Use of these in such a combination may give increased stability to an active ingredient. An L-amino acid is not particularly limited, and may be any one of glycine, cysteine, glutamic acid, and the like. A sugar is not particularly limited, and may be any one of the monosaccharides (such as glucose, mannose, galactose, and fructose), sugar alcohols (such as mannitol, inositol, and xylitol), disaccharides (such as sucrose, maltose, and lactose), polysaccharides (dextran, hydroxypropylstarch, chondroitin sulfate, and hyaluronic acid), derivatives thereof, and so on. A cellulose derivative is not particularly limited, and may be any one of methylcellulose, ethylcellulose, hydroxyethylcellulose, hydroxypropylcellulose, hydroxypropylmethylcellulose, sodium carboxymethylcellulose, and the like.

A surfactant is not particularly limited, and can be both an ionic surfactant and/or a non-ionic surfactant. As a surfactant, the following may be used: polyoxyethyleneglycol sorbitan alkyl ester base; polyoxyethylene alkyl ether base; sorbitan monoacyl ester base; or a fatty acid glyceride base.

As a buffer agent, the following may be used: boric acid; phosphoric acid; acetic acid; citric acid; ε-aminocaproic acid; glutamic acid; and/or a salt thereof, for example, an alkali metal salt and/or an alkaline earth metal salt, such as a sodium salt, a potassium salt, a calcium salt and a magnesium salt.

As an isotonizing agent, the following may be used: sodium chloride; potassium chloride; sugars; or glycerin.

As an isotonizing agent, the following may be used: sodium chloride; potassium chloride; sugars; or glycerin.

As a chelating agent, sodium edentate and citric acid may be used.

The medicaments and the pharmaceutical compositions according to the present invention can be used as solution preparations. Alternatively, they can be freeze-dried, so as to be in a good state for preservation. They can be used by dissolving them in water, a buffered solution containing saline, and the like, and then adjusting them to a suitable concentration, at the time of use.

The medicaments and the pharmaceutical compositions according to the present invention can be used as agents for preventing and/or treating a disease due to an abnormality in the function of a present protein and/or an abnormality in the expression of a present polynucleotide. In addition, the medicaments and the pharmaceutical compositions can be used in methods for preventing and/or treating the disease.

For abnormal symptoms due to an excess of a function of a protein according to the present invention, and/or the expression of a polynucleotide according to the present invention, an effective amount of an inhibitor that inhibits the function of a present protein and/or the expression of a present polynucleotide, may be administered to a subject together with a pharmaceutically acceptable carrier. The administration may result in obtaining an effect such as prevention, improvement, or treatment of the abnormal symptoms. Alternatively, the similar effect can be obtained by inhibiting the intrinsic expression of a present polynucleotide using an expression block method. The inhibition of the expression of a present polynucleotide can be achieved, for example, by using an anti-sense oligonucleotide, such as an oligonucleotide consisting of a partial sequence of a present polynucleotide. An oligonucleotide corresponding to a non-coding region of a present polynucleotide as well as an oligonucleotide corresponding to a coding region thereof, is useful as an anti-sense oligonucleotide used herein. In order to specifically inhibit the expression of a present polynucleotide, it is preferable to use a nucleotide sequence of a characteristic region of the polynucleotide.

A disease due to an abnormality in the function of a present protein and/or an abnormality in the expression of a present polynucleotide may be, for example, a tumor disease preferably exemplified by tonsil tumors and bronchial tumors. The tissue distribution of the polynucleotide shown by the nucleotide sequence set forth in SEQ ID NO: 1, which is a specific example of a polynucleotide according to the present invention, was found to be 2.77 times higher in a tonsil squamous cell carcinoma that is one of tonsil tumors, as compared to that in a normal tonsil tissue. Further, the expression of the polynucleotide was also found to be 1.65 times higher in a bronchial adenoid cystic carcinoma, as compared to that in a normal bronchial tissue. As described above, the protein encoded by the polynucleotide shown by the nucleotide sequence set forth in SEQ ID NO: 1 binds to a Rho family protein. Therefore, the protein can be considered to work as Rho-GEF. Among the Rho-GEF genes isolated so far, the following genes are known to relate to cancer: vav (Non-Patent References 3 and 4); ost (Non-Patent Reference 5); ibc (Non-Patent Reference 6) and the like. Thus, the present inventors believe that the high expression of the present polynucleotides relates to a tumor disease such as tonsil tumors and bronchial tumors. Further, the present inventors believe that the prevention and/or treatment of these diseases can be conducted by inhibiting the function of the present proteins and/or the expression of the present polynucleotides. The medicaments and the pharmaceutical compositions according to the present invention may be useful as agents for preventing and/or treating a tumor disease such as tonsil tumors and/or bronchial tumors. In addition, the medicaments and the pharmaceutical compositions may be used in methods of preventing and/or treating a tumor disease such as tonsil tumors and/or bronchial tumors.

Suitable dosage ranges of the medicament and the pharmaceutical composition according to the present invention are not particularly limited, and can be determined in accordance with the following: effectiveness of the ingredients contained therein; the administration form; the route of administration; the type of disease; the characteristics of the subject (e.g., body weight, age, symptomatic conditions, and whether a subject is taking other pharmaceutical agents); and the judgment of a physician in charge. In general, a suitable dosage may fall, for example, within a range of about 0.01 μg to 100 mg, per 1 kg of the body weight of the subject, and preferably within a range of about 0.1 μg to 1 mg, per 1 kg of body weight. However, the dosage may be altered using conventional experiments for optimization of a dosage that are well known in the art. The aforementioned dosage can be divided for administration once to several times a day. Alternatively, periodic administration once every few days or few weeks can be employed.

When administering the medicament or the pharmaceutical composition according to the present invention, the medicament or the pharmaceutical composition may be used alone, or may be used together with other compounds or medicaments useful for preventing and/or treating the target disease.

In terms of a route of administration, it may be either systemic administration or local administration. The route of administration that is appropriate for a particular disease, symptomatic condition, or other factors, should be selected. For example, parenteral administration including normal intravenous injection, intra-arterial administration, subcutaneous administration, intracutaneous administration, and intramuscular administration can be employed. Oral administration can be also employed. Further, transmucosal administration or dermal administration can be employed. In the case of use for tumor disease, it may be preferable to employ a direct administration into the tumor by injection, and the like.

In terms of an administration form, various forms can be selected in accordance with a treatment purpose. For example, a solid formulation may be employed such as a tablet, pill, powder, powdered drug, fine granule, granule, or a capsule. Alternatively, a liquid formulation can be employed such as an aqueous formulation, ethanol formulation, suspension, fat emulsion, liposome formulation, clathrate such as cyclodextin, syrup, or an elixir. These can be further classified, according to the administration route, into an oral formulation, parenteral formulation (drip injection formulation or injection formulation), nasal formulation, inhalant formulation, transvaginal formulation, suppositorial formulation, sublingual agents, eye drop formulation, ear drop formulation, ointment formulation, cream formulation, transdermal absorption formulation, transmucosal absorption formulation, and the like, which can be respectively blended, formed and prepared according to conventional methods.

(Diagnosing Method)

The proteins, polynucleotides, recombinant vectors, transformants, antibodies, or the compounds, which are provided in the present invention, can be used by themselves as a means for diagnosing a disease, such as a diagnostic marker or a diagnostic reagent.

According to the present invention, for example, use of all or a part of a polynucleotide according to the present invention allows the specific detection of the presence or absence of an abnormality in a polynucleotide or a gene containing the polynucleotide, or the presence or absence of expression thereof, in an individual, or in various kinds of tissues. The detection of a polynucleotide according to the present invention allows for a diagnosis of susceptibility to, onset of, and/or prognosis of, a disease due to an abnormality in the amount of a polynucleotide or a gene containing the polynucleotide, and/or, an abnormality in the function thereof.

Diagnosis of a disease can be carried out, for example, by detecting the presence of a polynucleotide according to the present invention, by determining the existing amount thereof, and/or by identifying a mutation, with respect to a sample to be tested (test sample). In comparison to a normal control sample, a change in the existence of a present polynucleotide, and a quantitative change thereof, can be detected. Alternatively, an amplified product obtained by amplifying a present polynucleotide using a known method may be subjected, for example, to the measurement of a change in size. In comparison to a normal genotype, a mutation such as deletion or insertion can be detected. Further, a polynucleotide amplified from a test sample may be subjected, for example, to hybridization to a labeled polynucleotide according to the present invention, which allows the isolation of a point mutation. The detection of such a mutation allows the aforementioned diagnosis.

The present invention can provide a qualitative or quantitative measurement method, for a polynucleotide according to the present invention, in a test sample. Further, a qualitative or quantitative measurement method for the mutation, in the specific region of a polynucleotide, can also be provided.

The tissue distribution of the polynucleotide shown by the nucleotide sequence set forth in SEQ ID NO: 1, was found to be 2.77 times higher in a tonsil squamous cell carcinoma that is one of tonsil tumors, as compared to that in a normal tonsil tissue. The tissue distribution was examined using BioExpress (GeneLogic), disease database information. Further, the expression of the polynucleotide was also found to be 1.65 times higher in a bronchial adenoid cystic carcinoma, as compared to that in a normal bronchial tissue. Meanwhile, as described in the above, the present inventors considered that the high expression of a present polynucleotide relates to a tumor disease such as tonsil tumors and bronchial tumors. Therefore, the detection of the increased amount of expression of the polynucleotide in a test sample allows execution of a method of determining whether the test sample is a test sample that is derived from a tumor disease such as tonsil tumors and bronchial tumors, or not. Such a determination method may also be included in the scope of the present invention. In this determination method, the increased amount of expression of the polynucleotide can be detected by comparing a test sample to a normal control sample. A human tonsil-derived tissue, or a human bronchia-derived tissue, may be preferably used as a test sample. A normal human tonsil-derived tissue, or a normal human bronchia-derived tissue, may be preferably used as a control sample. In the case that the amount of expression of the polynucleotide is increased in a sample derived from human tonsil tissue, compared to that in a control sample, preferably at least 2.5 times or more, more preferably at least 3 times or more, it can be determined that the test sample is a human tonsil tumor-derived sample. In the case that the amount of expression of the polynucleotide is increased in a sample derived from human bronchial tissue, compared to that in a control sample, preferably at least 1.5 times or more, more preferably at least 2 times or more, it can be determined that the test sample is a human bronchial tumor-derived sample. The phrase “amount of expression of the polynucleotide according to the present invention” means the amount of the transcription product of the polynucleotide.

A test sample is not limited to a human tonsil-derived tissue, or a human bronchia-derived tissue. Any tissues and cells derived from a living body can be used. Specifically, a sample derived from a living organism such as a cell, blood, urine, saliva, spinal fluid, biopsy tissue, or autopsy material, and the like, may be used as a test sample. As desired, a nucleic acid may be extracted from a test sample to prepare a nucleic acid sample for use. A nucleic acid may be any of a genomic DNA, RNA, and cDNA. A nucleic acid also may be enzymatically amplified by employing PCR, or other amplification methods. A nucleic acid sample may also be prepared according to various methods, for facilitating detection of a target sequence, for example, denaturation, digestion with restriction enzymes, electrophoresis, or dot blotting.

Any known gene detection methods can be used for detecting a polynucleotide according to the present invention or a gene containing the polynucleotide. Specifically, for example, plaque hybridization, colony hybridization, Southern blotting, Northern blotting, the NASBA method, reverse transcription-polymerase chain reaction (RT-PCR), or the like can be used. In addition, a gene detection method which allows cell level measurement, such as in situ RT-PCR, in situ hybridization, or the like, can be used for the detection. In such a gene detection method, it is useful to use an oligonucleotide, which consists of a partial sequence of a present polynucleotide, and has the property as a probe or a primer, for carrying out the isolation and/or the amplification of the polynucleotide, a gene containing the polynucleotide, or a mutant gene thereof. The phrase “oligonucleotide having the property as a probe” means an oligonucleotide that is capable of specifically hybridizing only to the present polynucleotide, and consists of a characteristic sequence of a present polynucleotide. The phrase “oligonucleotide having the property as a primer” means an oligonucleotide that is capable of specifically amplifying only a present polynucleotide, and consists of a characteristic sequence of a present polynucleotide. Further, when detecting a mutant gene capable of being amplified, a primer or a probe having a sequence with a predetermined length, which contains a mutation site within the gene, is prepared and used. A probe and a primer may have a nucleotide sequence consisting of, preferably from about 5 to 50 nucleotides, more preferably from about 10 to 35 nucleotides, and even more preferably from about 15 to 30 nucleotides. Specifically, an oligonucleotide shown by the nucleotide sequence set forth in SEQ ID NO: 5, or SEQ ID NO: 6, can be preferably used as a primer for amplifying a polynucleotide of the present invention or a fragment thereof, or as a probe for detecting a present polynucleotide. A labeled probe is normally used as the probe, but the unlabeled prove can also be used. Alternatively, the detection can also be carried out by measuring the specific binding to a ligand that was labeled directly or indirectly. Various methods are known for labeling a probe and a ligand. For example, nick translation, random priming, or a method utilizing kinase treatment, may be used. Labeling substances suitable for use include a radioactive isotope, biotin, a fluorescent substance, a chemiluminescent substance, an enzyme, an antibody, and the like.

PCR is preferable as a gene detection method, from the viewpoint of sensitivity. Any well-known method of PCR can be employed, as long as it is a method that uses a primer capable of specifically amplifying a polynucleotide according to the present invention, a gene containing the polynucleotide, or a mutant gene thereof. For example, RT-PCR may be employed. In addition, various modified PCT methods used in the art can be applied.

In addition to detection of a gene, PCR allows quantitative measurement of a polynucleotide according to the present invention, a gene containing the polynucleotide, or a mutant gene thereof. Such an assay method may be exemplified by a competitive assay, such as, an MSSA method, or PCR-SSCP, which is known as a mutation detection method that utilizes a change in mobility accompanying a structural change of a single-stranded DNA.

According to the present invention, for example, use of a protein according to the present invention allows the specific detection of, the presence or absence of, an abnormality in the protein itself, and in its function, in an individual or in various kinds of tissues. The detection of an abnormality in a protein according to the present invention, and in its function, allows a diagnosis of susceptibility to, onset of, and/or prognosis of, a disease due to an abnormality in the amount of the protein and/or an abnormality in its function.

The diagnosis of a disease by detecting a protein can be carried out, for example, by detecting the presence of a protein, by determining the existing amount thereof, and/or by identifying a mutation, with respect to a sample to be tested (test sample). That is to say, a protein according to the present invention, and/or its mutant, may be quantitatively or qualitatively measured. In comparison to a normal control sample, a change in the existence of a present protein, and a quantitative change thereof, can be detected. Alternatively, in comparison to a normal control sample, a mutation can be detected, for example, by determining an amino acid sequence. The detection of such a change or a mutation allows the aforementioned diagnosis. A test sample is not limited so long as the sample has the present protein and/or its mutant. A biological sample derived from a living organism, such as blood, serum, urine, biopsy tissue, and the like, may be used as a test sample.

A protein according to the present invention, and the protein with a mutation, can be measured using a protein according to the present invention, a fragment thereof, or an antibody against the protein or the fragment. Specifically, for example, the protein shown by the amino acid sequence set forth in SEQ ID NO: 2, a protein shown by the amino acid sequence having a deletion, substitution, insertion, or addition of one or several or more amino acids, in the amino acid sequence of the protein, the fragment thereof, or an antibody against the protein or the fragment, can be used.

Any protein detection methods, or protein quantitation methods which are well known in the art, can be used for quantitative or qualitative measurement of the protein. For example, the amino acid sequence analysis of a present protein allows a detection of a mutant protein. More preferably, an antibody (a polyclonal antibody or a monoclonal antibody) may be used for detecting the difference in the protein sequence, or the presence or absence of the protein.

The present invention includes a qualitative or quantitative measurement method for a present protein in a test sample, or a qualitative or quantitative measurement method for a mutation in the specific region of the protein.

Specifically, the aforementioned detection may be carried out by subjecting a test sample to immunoprecipitation, using a specific antibody raised against a present protein, and then analyzing the present protein by Western blotting or immunoblotting. Further, the detection of a present protein in a paraffin tissue section, or a frozen tissue section, may be carried out by means of immuno-histochemical techniques using a specific antibody raised against the present protein.

The preferable methods of detecting a present protein or its mutant, may be, for example, enzyme-linked immunosorvent assay (ELISA), radio immuno assay (RIA), immunoradiometric assay (IRMA), and immunoenzymometric assay (EMA), including a sandwich method using a monoclonal antibody and/or a polyclonal antibody. Alternatively, radio immuno assay, competitive binding assay and the like may be employed.

A protein, polynucleotide, recombinant vector, transformant, and/or antibody, which are provided according to the present invention, can each be used by themselves or in combination as a reagent, or the like. The reagent may be contained in a substance such as a buffer solution, a salt, a stabilizer, and/or an antiseptic agent, in addition to at least one member selected from a protein, a polynucleotide, a recombinant vector, a transformant, and an antibody which are provided according to the present invention. A known formulation means may be introduced, in accordance with the respective properties, at the time of formulation. The reagent can be used, for example, in a determination method, a method of identifying a compound, or a method of measuring the present protein, or a present polynucleotide, which is provided according to the present invention. In addition, the reagent is useful, for example, in elucidating an intracellular signal transduction pathway wherein a protein or a polynucleotide according to the present invention may be involved and the reagent can be used in fundamental research, such as research for a disease due to an abnormality of the protein or the polynucleotide.

The present invention further provides a reagent kit containing at least one member selected from a protein, a polynucleotide, a recombinant vector, a transformant, and an antibody which are provided according to the present invention. The kit may further contain a substance necessary for carrying out a measurement, such as a labeling substance, for detecting a protein or a polynucleotide according to the present invention, an agent for detecting the labeling substance, a reaction diluent, a standard antibody, a buffer solution, a washing agent, and a reaction terminating solution. As a labeling substance, the proteins described above, radioactive isotype, or the like, can be used. A labeling substance may be previously linked to the protein or the polynucleotide according to the present invention. The present reagent kit can be used, for example, in the determination method, the method of identifying a compound, or the method of measuring a present protein, or a present polynucleotide, which is provided according to the present invention. In addition, the present reagent kit can also be used as a test agent or a test kit in the test method using the aforementioned measurement method. The present reagent kit can further be used as a diagnostic agent, or a diagnostic kit in the diagnostic method using the aforementioned measurement method.

Hereinafter, the present invention may be explained more specifically with the following examples. However, the aspect of the present invention is not limited to these examples.

### EXAMPLE 1

### Gene Cloning

A human spleen-derived cDNA library was constructed in accordance with a method of Ohara et al. (Ohara, O. et al., DNA research, 1997, Vol. 4, p. 53-59). Specifically, double strand cDNA was synthesized by using a Superscript II reverse transcriptase kit (GIBCO BRL). At that time, an oligonucleotide (GACTAGTTCTAGATCGCGAGCGGCCGCCC(T)15: SEQ ID NO: 12, GIBCO BRL) which has a NotI site was used as a primer, and a human spleen mRNA (Clontech: catalogue number 6542-1) was used as a template. The cDNA was ligated to an adaptor (GIBCO BRL) which has a SalI site, and then digested with NotI, followed by electrophoresis with 1% concentration of a low melting point agarose to purify DNA fragments of 3 kb or more. The purified cDNA fragment was ligated with pBluescriptlI SK+ plasmid treated with SalI-Nol restriction enzyme. The recombinant plasmid was introduced into E. coli. ElectroMax DH10B strain (GIBCO BRL) by electroporation. Approximately 10,000 recombinants were selected from the constructed cDNA library to determine the both terminal DNA sequences of these clones. Among them, approximately 420 clones which contain novel genes were selected and determined for the entire nucleotide sequences of the cDNAs. Sequencing was carried out using DNA sequencer (RISA: SHIMADZU) and a reaction kit purchased from PE Applied Biosystem. The most of the sequences of shotgun clones was determined by using a dye terminator method (terminator labeling method).

The ORF was predicted for cDNA clones, whose entire nucleotide sequences were determined, by a conventional analysis method using a computer program. Subsequently, the ORF region was analyzed by a domain motif search, to identify a cDNA containing a region encoding a DH/PH domain that is an active domain of Rho-GEF.

As a result, cDNA having a novel nucleotide sequence, and containing a DH/PH domain coding region, was identified. Hereinafter, the cDNA is referred to as sh06537.

A sequence analysis showed that sh06537 consists of the nucleotide sequence set forth in SEQ ID) NO: 1 (5541 bp long), and encodes the amino acid sequence set forth in SEQ ID NO: 2 (1597 amino acids). A DH domain coding region of sh06537 corresponds to the region consisting of the nucleotides from the 3706^{th} nucleotide to the 4245^{th} nucleotide of the nucleotide sequence set forth in SEQ ID NO: 1. A PH domain coding region corresponds to the region consisting of the nucleotides from the 4345^{th} nucleotide to the 4680^{th} nucleotide of the nucleotide sequence set forth in SEQ ID NO: 1. In addition, a DH domain of the protein encoded by sh06537 corresponds to 180 amino acid resides from the 1178^{th} valine (al) to the 1357^{th} asparagine (Asn) of the amino acid sequence set forth in SEQ ID NO: 2. A PH domain corresponds to 112 amino acid resides from the 1391^{st} leucine (Leu) to the 1502^{nd} glutamic acid (Glu) of the amino acid sequence set forth in SEQ ID NO: 2.

### EXAMPLE 2

### DNA Expression and Purification

An expression vector for sh06537 that was isolated in Example 1 was prepared using GATEWAY™ Cloning Technology (Invitrogen). Then, the expression vector was used for expressing a protein that is encoded by sh06537, as FLAG-tagged protein, in 293EBNA cells (Invitrogen). In addition, an expression vector for DNA (hereinafter, may be referred to sh06537-D), which consists of a partial nucleotide sequence of sh06537 and lacks a DH/PH domain coding region, was prepared in the same manner. Then, the expression vector was used for expressing a protein, which consists of a partial amino acid sequence of a protein encoded by sh06537 and lacks a DH/PH domain, as FLAG-tagged protein, in 293EBNA cells, as well. The expression was confirmed by Western blotting.

Specifically, sh06537 that was isolated in Example 1 and inserted into the SalI-NotI site of pBluescriptII SK+, was used as a template for amplifying the ORF region, without a stop codon, of sh06537. Amplification was carried out by using pfu turbo (STRATAGENE). Oligonucleotides shown by the respective nucleotide sequences set forth in SEQ ID NO: 5 and SEQ ID NO: 6, were used as amplification primers. Then, an amplification product was inserted into pENTR/SD/D-TOPO in a reaction using TOPO cloning system to prepare an entry vector. The prepared entry vector was cleaved with HindIII/EcoRI to produce a gene fragment. On the other hand, sh06537 was cleaved with HindIII/EcoRI/ScaI to produce a gene fragment These gene fragments were ligated to each other, and introduced into competent cells (TOP10: Invitrogen). The transformed E. Coli cells were used for purifying DNA (herein after, may be referred to sh06537-F), by using a purification kit (QIAGENE). Sequencing analysis confirmed a sequence of the amplified region, and sequences upstream and downstream of the nucleotide sequence after treated with restriction enzymes, to be correct Sequencing reaction was carried out using DYEnamic ET Terminator Cycle Sequencing Kit (Amersham Biosciences). Electroporation and analysis were carried out using ABI PRISM 377. Next, sh06537-F was used together with a C-terminal 3×FLAG tagged protein-expression vector that was cleaved with BspEI for preparing a sh06537-F expression plasmid in a recombination reaction using LR clonase enzyme. Use of this expression plasmid allows the production of a protein encoded by sh06537-F, as a C-terminal 3×FLAG-tagged protein.

Next, an expression vector for sh06537-D was constructed using GATEWAY™ Cloning Technology (Invitrogen). Specifically, at first, sh06537, that was isolated in Example 1 and inserted into the SalI-NotI site of pBluescriptII SK+, was used as a template for amplifying a partial sequence of sh06537. The partial sequence of sh06537 consists of nucleotides from the 175^{th} nucleotide to the 3693^{rd} nucleotide of SEQ ID NO: 1 (i.e., SEQ ID NO: 3), and lacks a DH/PH coding region. The amplification was carried out using pfu turbo (STRATAGENE), and using oligonucleotides shown by the respective nucleotide sequences set forth in SEQ ID NO: 5 and SEQ ID NO: 7 as primers. Then, an amplification product was inserted into pENTR/SD/D-TOPO in a reaction using TOPO cloning system to prepare an entry vector. The prepared entry vector was cleaved with NaeI/EcoRI/PmaCI to produce a gene fragment. On the other hand, sh06537 obtained in Example 1 was cleaved with NaeI/EcoRI/SalI to produce a gene fragment. These gene fragments were ligated to each other and introduced into competent cells (TOP10: Invitrogen). The transformed E. Coli cells were used for purifying DNA using a purification kit (QIAGEN). Sequencing analysis confirmed a sequence of the amplified region and sequences upstream and downstream of the nucleotide sequence after treated with restriction enzymes, to be correct. Sequencing reaction was carried out using DYEnamic ET Terminator Cycle Sequencing Kit (Amersham Biosciences). Electroporation and analysis were carried out using ABI PRISM 377. Next, the purified DNA was used together with a C-terminal 3×FLAG-tagged protein-expression vector that was cleaved with BspEI, for preparing a sh0537-D expression plasmid, in a recombination reaction using LR clonase enzyme. Use of this expression plasmid allows the production of a protein encoded by sh06537-D as a C-terminal 3×FLAG-tagged protein.

The sh06537-F expression vector and the sh06537-D expression vector were respectively transfected into 293EBNA cells by lipofection. The 293EBNA cells had been seeded the day before transfection at 6×10⁴ cells/well in 24 well plates, and cultured in culture medium (IMDM medium (SIGMA), 10% fetal bovine serum, 4 mM glutamine, and 10 μg/ml gentamycin). On the next day, the transfection was carried out by means of using Lipofectamine™ 2000 (Invitrogen). Specifically, at first, each expression vector was added to serum-free DMEM (SIGMA), subsequently mixed with DMEM containing Lipofectamine™ 2000, and incubated at room temperature for 20 minutes. The obtained mixture was added to 293EBNA cells that had been seeded the day before, and cultured at 37° C. in the presence of 5% CO₂. The cells subjected to transfection were further cultured at 37° C. in the presence of 5% CO₂. On two days after transfection, the culture medium was removed from cultured cells to wash the cells with phosphate-buffered physiological saline (PBS). Then, the cells were lysed with lysis buffer containing 1% protease inhibitor cocktail (SIGMA) to prepare a cell lysate. The lysis buffer was composed of 25 mM Tris-HCl (pH 7.5), 150 mM NaCl, 1 mM CaCl₂, and 1% Triton X-100.

Each cell lysate was mixed with an equal volume of SDS-PAGE sample buffer, and subjected to heat treatment at 100° C. for 5 minutes, to prepare a sample for electrophoresis. SDS-polyacrylamide gel electrophoresis (SDS-PAGE) was carried out, and then the gel was equilibrated for 5 minutes or more, in blotting buffer. The proteins were transferred onto PVDF membrane. After blotting, the PVDF membrane was subjected to blocking at 4° C. for overnight in a solution (TBS-T+BA) containing the TBS-T mixed with Block Ace (Dainippon Pharmaceutical) at a ratio of 3:1. After blocking, the PVDF membrane was washed with TBS-T for 10 minutes or more with shaking. As a SDS-PAGE sample buffer, β-ME Sample Treatment for Tris SDS (Daiichi Pure Chemical s) was used. The running buffer for SDS PAGE was composed of 100 mM Tris, 192 mM glycine, 0.1% SDS, pH 8.3 (Bio Rad). The blotting buffer is composed of 25 mM Tris, 40 mM ε-amino-n-caproic acid, 20% methanol, and 0.05% SDS. The TBS-T is composed of 150 mM NaCl, 10 mM Tris-HCl (pH 7.5), and 0.05% Tween-20.

Anti-FLAG M2 monoclonal antibody (SIGMA) that was diluted to 1000-fold with TBS-T+BA was added to the PVDF membrane, and incubated at 37° C. for 1 hour or more. After that, the PVDF membrane was washed three times with TBS-T with shaking (20 minutes or more for each wash), and HRP-labeled goat anti-mouse IgG antibody (Cell Signaling Technology), that was diluted to 1000-fold with TBS-T+BA was added to the membrane. The membrane was incubated at 37° C. for 1 hour or more. Finally, the PVDF membrane was washed three times with TBS-T with shaking (20 minutes or more for each wash), followed by detection of the expressed protein that reacted with anti-FLAG antibody by using ECL Plus Western Blotting Detection System (Amersham biosciences). The chemiluminescence was visualized with detection device, Lumino Imaging Analyzer (TOYOBO).

The cell lysate prepared from the cells that were transfected with the sh06537-F expression vector showed a major band around 180 kDa. In addition, the cell lysate prepared from the cells that were transfected with the sh06537-D expression vector showed a major band around 130 kDa. The deduced molecular weight of a protein, which is expressed as a FLAG-tagged protein by the sh06537-F expression vector, is approximately 180 kDa. The deduced molecular weight of a protein, which is expressed as a FLAG-tagged protein by the sh06537-D expression vector, is approximately 180 kDa. On the other hand, none of these bands was detected in the cell lysate prepared from cells that were added with Lipofectamine™ 2000 without transfecting with the vector. Therefore, the major bands observed are believed to be a protein encoded by sh06537-F and a protein encoded by sh06537-D, respectively. Thus, the protein encoded by sh06537-F and the protein encoded by sh06537-D were obtained.

### EXAMPLE 3

### Detection of the Binding of a Protein Encoded by sh06537-F to a Rho Family Protein

The binding of a protein encoded by sh06537-F to a Rho family protein was examined by pull down assay, using the sh06537-F expression vector constructed in Example 2. In addition, the binding of a protein encoded by sh06537-D to a Rho family protein was examined in a same manner, using the expression vector constructed in Example 2, which contains sh06537-D that is DNA consisting of the partial sequence and lacking a DH/PH domain coding region.

RhoA was used as a Rho family protein. An expression vector for expressing RhoA as an N-terminal GST-fusion protein was prepared using GATEWAY™ Cloning Technology (Invitrogen). Specifically, RhoA gene was amplified by pfu turbo, using spleen first strand DNA of Multiple Tissue cDNA Panels (Clontech) as a template. An amplified product was inserted into pENTR/D in a reaction using TOPO cloning system to prepare an entry vector. Oligonucleotides shown by the respective nucleotide sequences set forth in SEQ ID NO: 10 and SEQ ID NO: 11 were used as primers for amplification reaction. The constructed entry vector was then subjected to a recombination reaction in the presence of LR clonase, using an N-terminal GST-fusion protein-expression vector, pDEST27, to prepare a GST-fusion Rho expression plasmid Sequencing analysis confirmed correct insertion of the nucleotide sequence of a coding region of each gene. Sequencing reaction was carried out using DYEnanmic ET Terminator Cycle Sequencing Kit (Amersham Biosciences). Electrophoresis and analysis were performed using ABI PRISM 377.

The sh06537-F expression vector and the RhoA gene expression vector were co-transfected into 293EBNA cells, for detecting the binding to the protein encoded by sh06537-F to a Rho family protein. Specifically, both expression vectors were added to serum-flee DMEM, subsequently mixed with serum-free DMEM containing Lipofectamine™ 2000, and incubated at room temperature for 20 minutes. The obtained mixture was added to 293EBNA cells. The 293EBNA cells had been seeded the day before transfection at 6.0×10⁴ cells/well in 24 well plates, and cultured at 37° C. overnight in the presence of 5% CO₂ prior to use in this Example. The cells subjected to transfection were cultured for 2 days at 37° C. in the presence of 5% CO₂. After culturing, the cells were washed with PBS, and then lysed with lysis buffer (see Example 2 for the composition) containing 1% protease inhibitor cocktail (SIGMA) to prepare a cell lysate. On the other hand, the sh06537-D expression vector was used, instead of the sh06537-F expression vector, for transfecting 293EBNA cells together with the RhoA gene expression vector. The obtained cells were used for preparing a cell lysate in a same manner as described in Example 2. A cell lysate prepared from cells transfected only with RhoA gene expression vector was used as a negative control.

Each cell lysate was subjected to a pull-down assay for detecting the binding of a protein encoded by sh06537-F, or a protein encoded by sh06537-D, to RhoA. 300 μL of each cell lysate, 20 μL of Glutathione Sepharose 4B in the lysis buffer, and 100 μL of the lysis buffer were mixed. Each sample was prepared in such a way that the final concentration of MgCl₂ and dithiothreitol (DTT) was 1 mM. After reacting at 4° C. for 1 hour with rotating by rotator, each sample was washed three times with 1 mL of cold lysis buffer (the final concentration of MgCl₂ was 1 mM) by centrifugation at 1,000 rpm for 15 seconds at 4° C. After washing and removing the supernatant, the Glutathione Sepharose 4B was added with 40 μL of a solution that was prepared by mixing SDS-PAGE sample buffer with an equal volume of the lysis buffer. After that, it was mixed with a mixer and subjected to a heat treatment at 100° C. for 5 minutes to prepare a sample for electrophoresis. SDS-polyacrylamide gel electrophoresis was carried out, and then the gel was equilibrated for 5 minutes or more in a blotting buffer. The proteins were transferred onto a PVDF membrane from the gel. After blotting, the PVDF membrane was subjected to blocking at 4° C. overnight in TBS-T+BA. After blocking, the PVDF membrane was washed with TBS-T with shaking for 10 minutes or more. The SDS-PAGE sample buffer, the blotting buffer, TBS-T and TBS-T+BA, which were used herein, had the same components with the respective buffers used in Example 2.

Anti-FLAG M2 monoclonal antibodies (SIGMA) that was diluted to 1000-fold with TBS-T+BA was added to the PVDF membrane, and incubated at 37° C. for 1 hour or more. After that, the PVDF membrane was washed three times with TBS-T with shaking (20 minutes or more for each wash), and then HRP-labeled anti-mouse IgG antibody (Cell Signaling Technology) that was diluted to 1000-fold with TBS-T+BA was added to the membrane. The membrane was incubated at 37° C. for 1 hour or more. Finally, the PVDF membrane was washed three times with TBS-T with shaking (20 minutes or more for each wash), followed by detection of the expressed protein that reacted with anti-FLAG antibodies by using the ECL Plus Western Blotting Detection System (Amersham biosciences). The chemiluminescence was visualized with a Lumino Imaging Analyzer (TOYOBO) detection device.

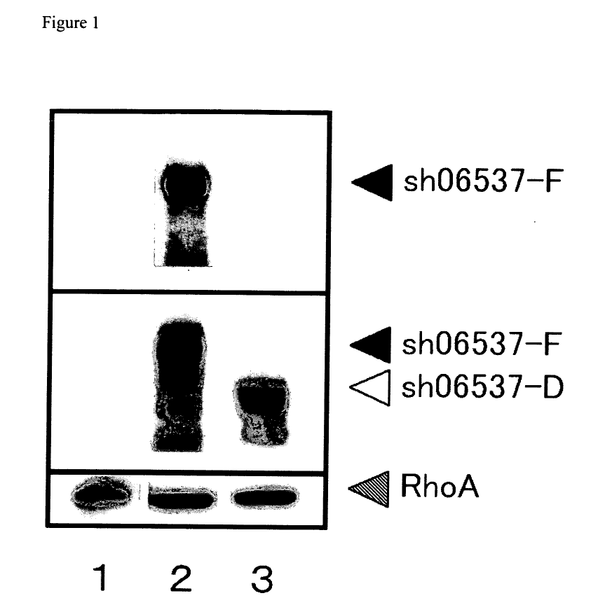

The results are shown in FIG. 1. A band which indicates the binding of a protein encoded by sh06537-F to RhoA was detected in a cell lysate prepared from the cells in which the protein encoded by sh06537-F was co-expressed with RhoA (lane 2 in the upper panel of FIG. 1). In contrast a band which indicates the binding of a protein encoded by sh06537-D to RhoA was not detected in a cell lysate prepared from the cells in which the protein encoded by sh06537-D was co-expressed with RhoA (lane 3 in the upper panel of FIG. 1). The expression of the protein encoded by sh06537-F or of the protein encoded by sh06537-D was observed in the cell (the middle panel of FIG. 1). The protein encoded by sh06537-F and the protein encoded by sh06537-D, which were contained in the respective cell lysates, were almost the same in amount (the middle panel of FIG. 1). In addition, the amount of RhoA contained in each cell lysate was almost the same (the lower panel of FIG. 1).

If the protein encoded by sh06537-F, or the protein encoded by sh06537-D, binds to RhoA, a band is detected at the position corresponding to the respective protein by using anti-FLAG antibody. The aforementioned results revealed that the protein encoded by sh06537-F binds to RhoA. The protein encoded by sh06537-D lacking a DH/PH domain coding region did not bind to RhoA. Thus, it was found that the binding of the protein encoded by sh06537-F to RhoA depends on a DH/PH domain of the protein. A DH/PH domain is an important domain participating in the Rho-GEF-mediated activation of a Rho family protein. In addition, it is considered to be an active domain of Rho-GEF. Therefore, the inventors believe that the protein encoded by sh06537-F binds to Rho family proteins such as RhoA, and thereby exhibits GEF activity.

### INDUSTRIAL APPLICABILITY

The protein, encoded by a polynucleotide according to the present invention, was bound to a Rho family protein, RhoA. The present proteins bind to a Rho family protein via a DH/PH domain. In general, Rho-GEF binds to a Rho family protein and activates the Rho family protein via a DH/PH domain. Therefore, it can be considered that the present proteins have a GEF activity for a Rho family protein. Use of the present proteins and polynucleotides allow elucidation and regulation of the Rho family protein mediated signal transduction pathway and cellular function. Further, use of the present proteins and polynucleotides allow for diagnosis, prevention and/or treatment of a disease due to an abnormal function of the present proteins and/or an abnormal expression of the present polynucleotides, for example, a tumor disease such as tonsil tumors and/or bronchial tumors. Thus, the present invention is extremely useful in a wide field including basic research and pharmaceutical development.

### GENERAL DESCRIPTION OF THE SEQUENCES

SEQ ID NO: 1: polynucleotide that encodes the protein (SEQ ID NO: 2) having a function as a guanine nucleotide exchange factor.

SEQ ID NO: 1: (3706): (4245) a region encoding a Dbl homology domain.

SEQ ID NO: 1: (4345): (4680) a region encoding a pleckstrin homology domain.

SEQ ID NO: 3: partial sequence of SEQ ID NO: 1 consisting of the nucleotides from the 175^{th} to the 3693^{rd}, which lacks a region encoding a Dbl homology domain and a pleckstrin homology domain.

SEQ ID NO: 3: polynucleotide encoding the amino acid sequence set forth in SEQ ID NO: 4.

SEQ ID NO: 5: designed oligonucleotide based on the sequence of SEQ ID NO: 1 for use as a primer.

SEQ ID NO: 6: designed oligonucleotide based on the sequence of SEQ ID NO: 1 for use as a primer.

SEQ ID NO: 7: designed oligonucleotide based on the sequence of SEQ ID NO: 1 for use as a primer.

SEQ ID NO: 8: polynucleotide encoding RhoA.

SEQ ID NO: 9: RhoA.

SEQ ID NO: 10: designed oligonucleotide based on the sequence of SEQ ID NO: 8 for use as a primer.

SEQ ID NO: 11: designed oligonucleotide based on the sequence of SEQ ID NO: 8 for use as a primer.

SEQ ID NO: 12: designed oligonucleotide for use as a primer.

1. A polynucleotide shown by the nucleotide sequence set forth in SEQ ID NO: 1 in the sequence listing or by the complementary nucleotide sequence, or a polynucleotide encoding a protein shown by the amino acid sequence set forth in SEQ ID NO: 2 in the sequence listing, or a polynucleotide shown by the complementary nucleotide sequence of the polynucleotide.

2. A polynucleotide selected from the following group, wherein the polynucleotide encodes a protein that binds to RhoA:
(i) a polynucleotide shown by a nucleotide sequence having a homology of at least 70% with the nucleotide sequence of the aforementioned polynucleotide,
(ii) a polynucleotide with a mutation or an induced mutation, such as deletion, substitution, addition of one or several nucleotides in the nucleotide sequence of the aforementioned polynucleotide, and
(iii) a polynucleotide that hybridizes to the aforementioned polynucleotide under stringent conditions.

3. A polynucleotide selected from the following group, wherein the polynucleotide encodes a protein having a guanine nucleotide exchange factor (GEF) activity for RhoA:
(i) a polynucleotide shown by a nucleotide sequence having a homology of at least 70% with the nucleotide sequence of the aforementioned polynucleotide,
(ii) a polynucleotide with a mutation or an induced mutation, such as deletion, substitution, addition of one or several nucleotides in the nucleotide sequence of the aforementioned polynucleotide, and
(iii) a polynucleotide that hybridizes to the aforementioned polynucleotide under stringent conditions.

4. A recombinant vector containing the polynucleotide according to claim 1.

5. A transformant that has been transfected with the recombinant vector according to claim 4.

6. The transformant according to claim 5, which has been transfected with the recombinant vector, and a recombinant vector containing a polynucleotide encoding RhoA.

7. A protein shown by the amino acid sequence set forth in SEQ ID NO: 2, in the sequence listing.

8. A protein encoded by the polynucleotide according to claim 2.

9. A method of producing the protein according to claim 7, comprising culturing a transformant that has been transfected with a recombinant vector containing a polynucleotide shown by the nucleotide sequence set forth in SEQ ID NO: 1 in the sequence listing or by the complementary nucleotide sequence, or a polynucleotide encoding a protein shown by the amino acid sequence set forth in SEQ ID NO: 2 in the sequence listing, or a polynucleotide shown by the complementary nucleotide sequence of the polynucleotide.

10. An antibody that recognizes the protein according to claim 7.

11. A method of identifying a compound that inhibits the function of the proteins according to claim 7, and/or the expression of a polynucleotide shown by the nucleotide sequence set forth in SEQ ID NO: 1 in the sequence listing or by the complementary nucleotide sequence, or a polynucleotide encoding a protein shown by the amino acid sequence set forth in SEQ ID NO: 2 in the sequence listing or a polynucleotide shown by the complementary nucleotide sequence of the polynucleotide, comprising detecting the presence, absence or change in the function and/or the expression under conditions where the interaction of a compound with the protein and/or the polynucleotide are allowed, and determining whether the compound inhibits the function of the protein and/or the expression of the polynucleotide.

12. The method according to claim 11, wherein the function of the protein is a guanine nucleotide exchange factor (GEF) activity for RhoA.

13. A method of determining whether a tissue specimen derived from a human tonsil tissue is a tissue derived from a human tonsil tumor or not, comprising measuring an amount of expression of the polynucleotide according to claim 1 in the tissue specimen.

14. The method according to claim 13, wherein the method determines that the tissue specimen is a tissue derived from a human tonsil tumor in the case when the amount of expression of the polynucleotide in the tissue specimen is 2.5 times higher than that in a control tissue derived from normal human tonsil.

15. A method of determining whether a tissue specimen derived from a human bronchial tissue is a tissue derived from a human bronchial tumor or not, comprising measuring an amount of expression of the polynucleotide according to claim 1 in the tissue specimen.

16. The method according to claim 15, wherein the method determines that the tissue specimen is a tissue derived from a human bronchial tumor in the case when the amount of expression of the polynucleotide according in the tissue specimen is 1.5 times higher than that in a control tissue derived from normal human bronchia.

17. An agent for preventing and/or treating tonsil tumors and/or bronchial tumors, comprising a compound that inhibits the function of the protein according to claim 7 and/or a compound that inhibits the expression of a polynucleotide shown by the nucleotide sequence set forth in SEQ ID NO: 1 in the sequence listing or by the complementary nucleotide sequence, or a polynucleotide encoding a protein shown by the amino acid sequence set forth in SEQ ID NO: 2 in the sequence listing, or a polynucleotide shown by the complementary nucleotide sequence of the polynucleotide, as an active ingredient.

18. A method of preventing and/or treating tonsil tumors and/or bronchial tumors, comprising using a compound that inhibits the function of the protein according to claim 7 and/or a compound that inhibits the expression of a polynucleotide shown by the nucleotide sequence set forth in SEQ ID NO: 1 in the sequence listing or by the complementary nucleotide sequence or a polynucleotide encoding a protein shown by the amino acid sequence set forth in SEQ ID NO: 2 in the sequence listing, or a polynucleotide shown by the complementary nucleotide sequence of the polynucleotide.

19. A reagent kit containing at least one selected from the protein according to claim 7, a polynucleotide shown by the nucleotide sequence set forth in SEQ ID NO: 1 in the sequence listing or by the complementary nucleotide sequence, or a polynucleotide encoding a protein shown by the amino acid sequence set forth in SEQ ID NO: 2 in the sequence listing or a polynucleotide shown by the complementary nucleotide sequence of the polynucleotide, the recombinant vector containing the polynucleotide, the transformant that has been transfected with the recombinant vector, and the antibody that recognizes the protein.
